(19) United States
(12) Patent Application Publication
(10) Pub. No.: US 2004/0143297 A1
(43) Pub. Date: Jul. 22, 2004

(54) Advanced automatic external defibrillator powered by alternative and optionally multiple electrical power sources and a new business method for single use AED distribution and refurbishment

(76) Inventor: Maynard Ramsey III, Tampa, FL (US)

Correspondence Address: Maynard Ramsey III, 903 Golfview St., Tampa, FL, 33629, US

(21) Appl. No.: 10/348,564

(22) Filed: Jan. 21, 2003

(51) Int. Cl. A61N 1/39

(52) U.S. Cl. 607/5

(57) ABSTRACT

An AED being powered by 120/240 VAC electrical power alone, being powered by external DC power alone, or any in combination with or without internal-integral battery power, and further an AED access service business method for sales of access to AEDs. The inventive AED, in addition to the defibrillator circuitry comprises a long, tangle free power access cord to be plugged into an external source of AC or DC power and optionally, additional sets of body surface and alternative electrodes positioned in the esophagus and/or heart. The AED has additional advanced capabilities including the ability to deliver rapid sequential shocks through one or more sets of patient electrodes, and the optional mode of shock delivery whereby the shock is delayed while the AED continues to analyze the patients ECG waveform and delays the defibrillation shock or sequence of shocks until the ECG analysis indicates conditions are optimum for successful defibrillation.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### FIELD OF INVENTION

The present invention relates generally to automated external defibrillators (AEDs) and the business method of providing them to the public. More specifically it relates to an AED with multiple substantial improvements over current AEDs, such improvements being in: powering reliability and versatility, defibrillation efficacy, tampering and environmental contamination protection, extended shelf life, affordability, ease of ownership and maintenance, and visual aesthetics. As a part of my invention, a new AED distribution business model and method is developed.

Such multiple substantial improvements over current AEDs comprising:

Powering Reliability and Versatility

Currently all AEDs are powered by internal-integral DC voltage batteries, said AED batteries being user replaceable in the field and internal to, or integral with, the main AED enclosure. (For purposes of this document, by the description of current AED's battery or batteries as being internal-integral is meant those AED battery or batteries that are contained within the AED enclosure itself or are inserted into a battery pack receptacle or compartment in the AED enclosure and are snapped or locked in place so that while powering the AED, they become an integral or internal part of the AED unit and are not connected to the AED by external wires. These internal-integral batteries are also designed to be replaced by the user in the field. From hence forth in the patent application the word internal-integral is used for such batteries as are used for powering all current AEDs and as described above.) For reliability, versatility, and reduced costs, my inventive AED is connected to and powered by at least one of the following:

1) exclusively by ordinary 110 or 220 volts alternating electrical current with no batteries of any kind being used,

2) primarily by ordinary 110 or 220 volts alternating electrical current, where no essential defibrillation functions are powered by batteries,

3) exclusively from external, non-integral sources of DC power, such as generators or batteries found in portable battery packs, motor vehicles, aircraft, and water vessels,

4) dual potential external sources of power, both AC and DC, whereby my AED is automatically powered by a factory set, or user selected, preferential source if both AC and DC sources are connected simultaneously, and otherwise the AED uses the available source if only one source is connected, and

5) internal-integral batteries plus one or more of external power sources, such external power sources being either AC or DC or both and with automatic priority selection of the power source actually used by the AED if any or all are power sources are available,

6) internal-integral batteries plus power connections for external power sources, plus an emergency human powered power generator for use when all other power sources are unavailable

7) a replacement power supply module for the traditional battery pack found in all current AEDs, wherein the replacement module is physically shaped like the normally present battery pack, but where the replacement module is an alternative power supply that is powered from external AC or DC electrical power thus permitting an existing AED to be powered by external power sources

Defibrillation Efficacy

Currently all AEDs use two surface electrodes for defibrillation. My AED provides the option of using more than two surface electrodes as well as using optional cardiac and esophageal defibrillation electrodes, all such additional electrodes are utilized for improving defibrillation efficacy when needed in difficult cases. Similarly, my AED is so designed and constructed so as to allow multiple shocks to be delivered rapidly where in such multiple shocks utilize the same set of electrodes, or optionally a different set, on each successive shock of a multiple shock sequence. Additionally, to further enhance defibrillation efficacy, my new AED can, after detecting ventricular fibrillation (VF), optionally delay defibrillation shocks until the moment when such shock has the highest likelihood of being effective, such moment being determined by the rate and or the morphology of the VF waveform. All of the above enhancements of efficacy in my AED have been demonstrated experimentally to increase the effectiveness of defibrillation, but have never before been incorporated into an AED to increase its effectiveness.

Tampering and Environmental Protection

The inventive AED is optionally equipped with a method of sealing the AED enclosure to prevent environmental contamination, such sealing of the AED enclosure being accomplished by at least one of: 1) using a gasket seal between the lid and the body of the enclosure, 2) using an external circumferential tape seal to seal the gap between the AED enclosure lid and the body of the AED enclosure, or 3) by providing the AED with a full or partial protective cover which is sealed. All of these sealing methods are designed to prevent or reveal tampering and to protect against environmental assaults such as water, moisture, dust, dirt, and corrosive gases.

Shelf Life

The inventive AED provides a minimum of 4 year maintenance free shelf life (as compared to the traditional 2 year shelf life of existing AEDs), since it is powered by at least one external source of power (versus internal-integral batteries only) and is sealed for protection. In one embodiment, a small, non-essential battery is used to keep time and power a suitable annunciator when the shelf life has expired. In another embodiment, internal-integral power batteries with four year shelf life are present, but in the event of their failure, external power sources are usable for powering the AED and hence life saving can be accomplished even if the internal-integral batteries are dead.

Affordability and Reliability

The inventive AED is less expensive than current AEDs because in the preferred embodiment, it is designed for single use, contains no power batteries, and is refurbishable at the factory at a price less than one half of the original new purchase price.

Ease of Ownership and Maintenance

The inventive AED is easier to own and use because in the preferred embodiment, the unit is single use, factory sealed, and there are no batteries to test or replace, thus eliminating the field maintenance of batteries and electrodes required with all existing AEDs.

Visual Aesthetics

The inventive AED is designed such that the various surfaces of the AED enclosure, or its protective cover, may be optionally decorated in a way that permits the user to select the degree of visual presence obviousness while the AED is stored and awaiting use. This external decoration or indicia provides the AED an aesthetic presentation while it is awaiting use and allows it stored for quick access on a table or shelf in the home or office.

New AED Distribution Business Model and Method

The new AED business model obsoletes the current method of AED distribution. It changes the nature of the AED business from an “AED for defibrillation” product business to a “single defibrillation with an AED” service business. In this new business model, single use defibrillators are acquired by purchase or rental for 4 or more years and then, when used or expired, are disposed of, or alternatively are refurbished by the factory at a cost which is less than one half the original purchase cost. This model is linked tightly with the long shelf life, low cost, single use inventive AED, the combination of which is designed to enable a service which provides for the placement of a single use AED for emergency defibrillation in every home and workplace, where the cost of such AED placement service is less than cable TV service or home security monitoring service.

This new business method or business model is completely different from that model used currently to promote and sell the traditional battery powered, multiple use, user refurbishable, AED designs. All of these current AEDs require refurbishment by the user or owner after each use and before being placed back into service without any testing or validation by the manufacturer. My new AED business method substantially reduces the technical complexity and the cost to the user for the ready availability of an AED and therefore greatly broadens the population both financially able to purchase or rent an AED and technically competent to make sure it is maintained and ready for use. This new business approach hence broadens the availability of AEDs to include placement in all homes and workplaces as well as in all work vehicles. Overall, the new business method here disclosed has been invented to create a new method of doing AED marketing and distribution, treating the distribution of AEDs more as a service than the traditional product sales business in which the user is responsible for purchasing and maintaining themselves the AED. This new business approach will create the establishment of single use “Personal AEDs”, the use of which is principally described as “Private Access” defibrillation in that the subscriber to the AED service or their family or work associates will be the ones to utilize the device in an SCA emergency. This new inventive approach is in complete contrast to the existing AED business model of selling multi-use, user refurbishable AEDs to various agencies for “Public Access” defibrillation. My new AED business method which enables and promotes “Personal AEDs” for “Private Access” will eventually save many more lives at less cost than the “Public Access” AEDs currently being deployed. The greater saving of life by single-use, “Personal AEDs” being kept in the home and workplace as compared to multi-use “Agency AEDs” in public places is because approximately 80% of sudden cardiac arrests (SCAs) occur in a private setting, such as the home or workplace, as compared to the 15-17% in public settings such as malls, airports, and stadiums. This new business model, selling the single use of an AED for defibrillation, transforms the traditional AED product business into what is mainly an AED service business, where the user buys the AED service initially for the price of a low cost AED. That purchased AED is good for one use or 4 or more years shelf life, after either one of which, the subscriber pays less than half of that initial amount for factory refurbishment of said same AED. Or alternatively, since all of such AEDs are alike, the manufactures may ship to the subscriber a refurbished AED of like manufacture upon notification or upon expiration of shelf life, before the original AED is taken out of service, thus assuring the subscriber is never without their AED. The motivation for this new business model is to reduce the cost and complexity of having a life saving AED in the home and workplace, and hence to greatly expand the access to this life saving technology to ordinary people in private places.

Since one the preferred embodiments of the inventive AED uses 110 or 220 volts of alternating electrical current power (120/240 VAC), it is to be understood clearly that said AC power is the ordinary 110 or 220 volts of alternating electrical current power found in all homes and workplaces and which is readily accessible with an ordinary wall power outlet into which is plugged a power cord with a two or three pronged mating electrical plug. In the context of this AED invention, such alternating electrical current which powers several of the embodiments of the inventive AED will often be described as 120/240 VAC electrical current reflecting that in some countries the standard mains or line power is between 100-120 VAC while in others the standard alternating current electrical power in use is 200-260 VAC. When the terms AC, 120/240 VAC, line, or mains power is used in this document, it is meant to include all such electrical power utility alternating currents as used in the various countries throughout the world. Conversely, when DC power is referred to it is understood that it is direct current electrical power supplied by some form of battery (internal-integral batteries or external batteries) or by an external DC power generator or AC to DC power converter, and when motor vehicle DC power is referred to it is understood that it is low voltage DC, typically 12-24 volts and supplied by the vehicle's battery or generator.

### BACKGROUND OF THE INVENTION

It can be appreciated that automated external defibrillators (AEDs) have been in use for at least 20 years, treating a very small segment of the estimated 450,000 annual victims of sudden cardiac arrest (SCA). Typically, AEDs are used in hospitals, EMS vehicles, and in the last 5-10 years, in public places such as airports, airplanes, shopping malls, supermarkets, schools, and the like for public use in terminating ventricular fibrillation (VF) or ventricular tachycardia (VT). Such cardiac arrhythmias are life threatening since they do not provide a coordinated heart contraction capable of pumping blood effectively and hence of sustaining consciousness and ultimately life itself. Specifically, VF will result in brain damage if the SCA victim's heart is not converted back to a more normal rhythm within 3-6 minutes of initial cardiac arrest, and ultimately, death in a matter of a few additional minutes. For practical purposes, this means that a rescuer must apply the defibrillator within the first 2-4 minutes after a cardiac arrest if the patient is to have a good chance of survival without any brain injury. The defibrillator used may be either the totally manual type as is often used in hospitals and in emergency medical service (EMS) vehicles by highly trained personnel, or a defibrillator of the automatic type, such as the AEDs now being placed in public places for public access and use by untrained good Samaritans. All of the AEDs now in use are approximately equivalent in function and have been for many years. They are simple to use and present no real difficulty in proper use even by untrained persons, including pre-teen age children. Though there are many patents issued each year in the AED field, there have been no major improvements to the AED technology for many years, it being understood that “major improvements” are improvements to AEDs that truly result in greater AED access, greater reliability, greater defibrillation success, greater willingness by individuals or agencies to acquire them and thus resulting in more lives being saved ay AEDs. My invention is a major improvement in the art of AEDs and their proliferation and hence availability in that it provides an AED with lower cost, more reliable, greater defibrillation efficacy, and greater shelf-life AED and which is coupled with a new business model designed to decrease costs and greatly increase private access to defibrillation. This new AED and business model provides these major improvements by offering a 5 year maintenance free, single use AED with factory refurbishment, thus supplying a true AED service versus simply selling AEDs to customers as is currently practiced by all manufacturers and distributors.

The present invention is a substantial improvement to all of the existing AEDs which are multi-use, user refurbishable, battery powered (both rechargeable and disposable battery types). My new design AED uses no batteries at all (or in one embodiment, batteries only for non-essential functions like a reminder that the shelf life is expired and in another embodiment, an external battery for power) and is designed principally for single patient use and is therefore more reliable and less expensive to produce, sell, and maintain. The single use design and the accompanying new business method of single use sale and factory refurbishment substantially improves the affordability and accessibility of the “Personal AED” for “Private Access” defibrillation. Such home and workplace location of personal AEDs is absolutely essential for saving many of the large numbers of patients who have an SCA in such locations since it will allow much more rapid AED application and use in treating the majority of cardiac arrests victims as compared to the response time of most EMS vehicles and personnel. This new AED, integrated with the new AED business method, will enable the broad deployment of “Private Access” AEDs in the same way that fire extinguishers and fire detectors are privately purchased and utilized in the home today by individuals who want to be prepared for and survive a fire, even if the likelihood of fire in any individual home is low. The FDA has recognized the benefit of home placement of AEDs and this approved AED placement in the home is already practiced on a very limited basis. The numbers of AEDs in the home are very limited due to the reliability, shelf life (typically 2 years), and maintenance issues specifically associated with the existing AEDs exclusive use of internal-integral batteries for all of its functions, as well as the overall costs associated with current AED acquisition, refurbishment, and maintenance.

As suggested above, the chance of successful defibrillation is greatly diminished as the duration of time increases between the cardiac arrest and the time of defibrillation. Studies in academic institutions have shown that the chance of successful patient resuscitation and injury free survival of the patient decreases approximately 10% for each minute from the time of onset of cardiac fibrillation and the successful defibrillation of the patient. Broken down into seconds, the patients chances for survival are decreasing approximately 1% every 6 seconds.

If one assumes that brain injury free survival is the real goal, then only approximately 4 minutes are allowed. Literally, every second counts and if defibrillation is attempted within the first minute or two after fibrillation begins, it is virtually 100% successful. The very short interval during which the patient can almost always be defibrillated, and can be saved without likely brain damage, clearly creates an imperative for the “fastest possible time” to defibrillation if victims of SCA are to be saved without permanent brain injury. Any delay can potentially make the difference between complete recovery and brain damage and death. Since approximately 80% of SCAs occur in the home, and since it takes EMS typically 10-15 minutes to arrive on the scene and use a defibrillator of either the manual or AED type, most all home based SCA victims die or have brain damage and survival after an SCA in the home is only 2-4%. Personal AEDs, and the rapid access by family and friends that their possession close at hand implies, can change this currently awful survival rate dramatically since close to 100% of SCA victims can be defibrillated if the AED, or other defibrillator type, is applied within 1-2 minutes.

In light of these data, it is understandable that the reduction of time between cardiac arrest and defibrillation has been a major public health imperative for the past 10-15 years, and the public health and corporate agency's approach has been “public access” AEDs. Laws have been enacted in some states that require the placement of AEDs in all schools in that state. Clearly this is desirable from a public health perspective, but the cost of each AED is typically between $1,200 and $4,000. However, these costs present a financial burden on any agency or person wanting to provide AEDs for treatment of ventricular fibrillation (VF) in cases of SCA. Thus, the cost of such AEDs prevent their acquisition by many agencies and businesses, and most importantly, prevent their acquisition by most families for placement in their homes. The aim of my invention is to create an AED low enough in cost, and with 5 year maintenance free reliability, which when provided through the new single use with factory refurbishment (SUFR) business model, will provide affordable private access to AEDs. Such AEDs will prevent this needles waste of life by having this affordable, reliable lifesaving device located in virtually all homes and workplaces.

The placement of AEDs in public places for bystander use on a patient suffering from cardiac arrest has been shown to be effective in reducing the time to defibrillation and in increasing survival (50% survival) as compared to the traditional waiting for the EMS paramedics to arrive and attempt defibrillation (10% overall survival, worse if in the home). Unfortunately, the impact of this greater public availability of AEDs on the overall survival of SCA vicitms has been only minimally impacted, since it is estimated that approximately 80% of cardiac arrests occur privately in the home. Clearly this argues for the broad deployment of AEDs in the home and workplace. Additionally, since it has been shown that untrained 12 year children, as well as untrained adults, are both capable of applying the AED to a fallen person successfully, the old concept that training must be supplied in all cases before a person can use the modern AED is fallacious and is being discarded as a “must have” before deploying AEDs into the field for use by untrained bystanders, and is important in removing psychological impediments to “Private defibrillation” using “Personal defibrillators”. It is an object of my invention to provide a low-cost, maintenance free, long shelf-life AED and also a new AED business process for dramatically increasing the availability and hence the successful deployment of AEDs into homes and workplaces.

### PRIOR ART AEDS

AEDs have been in use for over 15 years. Existing AEDs are all basically alike in design and operation and are produced by many companies. They all have a pair of self-sticking pad electrodes that are attached to the chest of the patient for ECG monitoring and shock delivery, a central processor that analyzes the patients heart rhythm from the ECG and determines if a shock is needed, and if needed, all of these current AEDs deliver a biphasic shock of 150 J to 360 J of energy to the patient through the two body surface electrodes. Many base the duration and strength of the shock on the impedance of the patient as judged by measuring the resistance to current flow through the patient using the surface electrodes attached to the patient. Though there are many patents on various features of AEDs, all current AEDs are similarly designed and there is very little real difference in them as a practical matter; all seem approximately equally effective when they are functioning properly, have good batteries, and are used properly, and equally important, when they are used within the first 2-3 minutes after sudden cardiac arrest (SCA). In summary, all existing AEDs do the same thing in basically the same way, and even generally look the same, typically being a bright color for high visibility and rugged construction since they are all designed for multiple uses without factory refurbishment.

The Top Source of Problems with all Existing AEDs is . . . Internal-Integral Batteries

The most fundamental similarity, and problem, in all of the prior art AEDs is the design approach in which all prior art AEDs contain at least one or more internal-integral batteries as their sole and exclusive source of power for all functions, both essential functions like ECG analysis and the creation and delivery of the defibrillation shock, and non-essential functions like continuously self testing the unit and the batteries, which actually then runs down the batteries. The batteries may be rechargeable or non-rechargeable, but all existing designs of AEDs rely solely on internal-integral batteries to supply the energy for powering the ECG monitoring and analysis functions, determination of the need and the delivery of defibrillation shocks, as well as the creation of the high voltage energy that is stored in capacitors and which is discharged into the patient in an attempt to shock the heart back to a more normal rhythm which will pump enough blood to prevent brain damage or death, keeping time.

Despite the often poor reliability, the known maintenance issues, and the cost of replacement, the use of integral battery power as the main energy source for all existing AEDs is actually beneficial in many circumstances, particularly in “Public Access” AEDs. This use of internal-integral batteries in current AEDs allows great portability of the AED thus enabling defibrillation even when far away from other, more reliable, power sources such as the ubiquitous household power outlet for 120/240 VAC power or the reliable external DC power from the cigarette lighter socket found in most motorized vehicles and most power boats. However, in the public access defibrillation mission, the portability and elimination of the need for alternative power provided by using integral AED batteries is very important to the primary users of AEDs today, such as EMS personnel and bystanders, allowing defibrillation to occur in locations where there is no other energy source readily accessible.

Though they can create this sometimes essential portable power, the problems with internal-integral AED batteries are many. Dead or weak AED batteries, due to age, internal defects, or multiple prior uses without subsequent testing or replacement, can render an AEDs totally useless in the crucial moments when it is greatly needed to actually save an SCA victims life.

In a less dramatic form of battery malfunction, the battery may simply be weak from age or prior use and hence is very slow in charging the high energy storage capacitors prior to shock delivery; such charging must be done each time before the AED can deliver a shock to defibrillate the patient. This delay in charging from a weak battery is unacceptable when every second counts. This time to charge using weak batteries can exceed the AAMI/AHA recommended 8-10 seconds desirable charge time and stretch to as long as 30 seconds with batteries that are very weak. If three shocks are required, such weak batteries can result in brain damage by consuming 90 seconds of valuable time waiting for the completion of charging. A charge time of 2-4 seconds, or even less for rapid multiple, sequential shocks is highly desirable, and my inventive AED is capable of doing this rapid charging, even repeatedly, because it utilizes ordinary 120/240 VAC power or alternative large external batteries such as engine starting batteries found in all motor vehicles and power boats. These alternative sources of power are unusable by current AEDs, but are highly advantageous since they do not run down when used as the source of AED power as compared to the current AED's integral battery, which does run down and eventually dies completely.

This presence of internal-integral AED batteries also leads to other AED problems such as limited shelf life, since all batteries discharge with the passage of time even if they are not actually being used to save lives, and generally two years is the recommended interval of battery replacement even if not used. This two year battery shelf life, (as well as the typical two year shelf life of electrodes), limits the shelf life of current AEDs to approximately two years. Additionally, since batteries powerful enough to supply the high energy shocks required are relatively heavy, they increase the weight and size of AEDs. The use of internal-integral batteries as the sole source of power in all existing AEDs also requires additional electronics to create the high voltage needed for defibrillation from the low voltage DC batteries, such additional electronics further increasing costs and weight.

However, the most dramatic problem with internal-integral AED batteries is exceptionally alarming. Since these batteries are small electrochemical energy plants, which produce a limited amount of electricity used to power the AED, if there is a battery malfunction there can be excessive heat generated, essentially an electrochemical plant meltdown, with consequent destruction of the battery itself and potential damage to the rest of the AED, rendering the AED useless even with a new battery. In extreme cases, instances of which have been reported to the FDA, the AED's battery can actually explode and injure the user as well as destroying the AED itself.

This multitude of undesirable characteristics of batteries are well known to manufacturers of AEDs, and have prompted AED manufacturers to create battery maintenance requirements that are to be performed by the user or owner on a regular and scheduled basis. The purpose of this routine maintenance is to reduce the likelihood of battery explosions or premature or unknown battery exhaustion, any of which will render the AED useless for defibrillation and potentially hazardous to the operator as well.

However, these battery maintenance requirements of existing AEDs must be scheduled and their performance tracked; further, they are time consuming, relatively complicated, and require some technical knowledge and even in some cases mathematical calculation to accurately track the remaining life in the battery as time passes. Hence, these maintenance requirements are, as a practical matter, not achievable by some EMS professionals and surely not achievable by most lay persons who would like to have access to a “Personal AED” in their home or workplace. Hence, the lay person who purchases a current AED for his or her home or workplace may not know, at any given time, the maintenance status of their AED's internal-integral batteries and must therefore test the device to know for sure their status, using a test method usually built into the AED. However, the requirement to perform such a test must be remembered by the user and the testing takes time and also uses up some of the battery capacity during the test. Many AEDs are designed to have automatic self tests run periodically, which is convenient and can provide a warning that battery life is low; however, these self tests also consume some of the battery's capacity, consequently reducing the shelf life of the battery and consequently of the shelf life of the AED itself. However, if the battery has actually failed completely, the self test will not work at all, and if the user is not very familiar with the operation and maintenance of the AED, they will not be aware of the presence of a dead battery.

Even More Problems with Internal-Integral Battery Power in Existing AEDs

An additional problem with the batteries used in most AEDs is that they are special designs and special shapes designed to mate with a specific AED from a single manufacturer. Obviously, such specialized batteries are not available except from the manufacturer and hence are not readily available when replacement is needed. Also, they are expensive, often costing as much as $100-$200 each. This excessive cost results in a reluctance for users to routinely replace them even when not used, and hence, there will be times when the AED is needed, but the battery is dead or very weak, and no replacement is readily available. The lack of a functioning AED internal-integral battery is a truly fatal deficiency in the case of the need to assist a person with SCA, since it is to be clearly understood, that when a person has ventricular fibrillation (VF) there is no treatment that will restore an effective heart beat other than defibrillation with a high power shock from a defibrillator.

These well known problems with batteries prompt most professional EMS personnel to keep an extra AED battery, or two, or three, with them or in their vehicle at all times. All of these problems with batteries are eliminated in my 120/240 VAC or external DC battery powered AED which in several embodiments contains no internal-integral batteries at all, and in one embodiment, only a small battery for non-essential functions such as keeping time and shelf life expiration annunciation. Hence, in several embodiments of my new AED, there are no internal-integral batteries to replace or to test or to worry about, or most importantly, to fail and prevent proper functioning when the AED is needed.

Internal-Integral Battery Power also Increases Costs

The battery powered design of all existing AEDs also results in additional manufacturing expense which my present invention avoids. This additional expense incurred by an AED design that uses one or more internal-integral batteries for its power consists of both the cost of the large powerful battery itself, but also for the additional circuitry which is required to convert the relative low voltage of the battery to the high voltage energy required for successful defibrillation. Similarly, as mentioned above, replacement batteries cost significantly, and since all batteries have a finite shelf life, an AED's battery must be replaced periodically even if not actually used for defibrillation. If the AED is actually used, the battery must be replaced more often. One currently available AED design uses ten small replaceable batteries which, when weak, must all be replaced. Such a multi-battery design also introduces additional reliability issues over those of single internal-integral battery AED design, since there are many more mechanical contacts associated with mounting all ten batteries as compared to designs that use a single large battery. Such mechanical contacts are often a source of failure in electronic devices of any kind and having twenty of them increases the probability of a contact failure.

Battery Rechargability is not an Answer

Some AED batteries are rechargeable, which can help reduce costs if the unit is used frequently as might be the case of an AED used by EMS vehicles. However, the rechargability of these batteries introduces new, undesirable variables such as the need and cost for a charger, and the need to recharge the batteries frequently whether used or not, since rechargeable batteries typically have a “self-discharge” rate much greater than non-rechargeable batteries. Many rechargeable batteries also have a well known “memory effect” that reduces battery capacity, often to unknown levels, if not properly conditioned periodically by three complete charge and discharge cycles, difficult for trained EMS professionals, but a true impracticality for an individual with a personal AED in their home.

The design of the AED to make the internal-integral power battery easily user replaceable also increases cost and mechanical/electrical complexity for the overall AED. These increased design and manufacturing costs are ultimately reflected in a higher cost per use of the AED and are totally eliminated by my new AED which either uses external power exclusively or non-user-replaceable internal-integral batteries in addition to the ability to use external power sources. Thus, in my AED, if the internal-integral batteries fail, the user simply plugs the AED into the wall power socket or into the cigarette lighter of a vehicle to power the unit. In AEDs placed in the home however, there is usually little need for the additional expense of internal-integral batteries since 120/240 VAC is always at hand.

Reliance on Internal-Integral Batteries for Power is not the Only Problem with Current AEDs

There are other problems with existing AEDs in addition to the problems associated with using batteries as their energy source. Current AEDs are also designed for high visibility in public places or EMS vehicles and as such, these AEDs look ugly in a person's home or workplace if visibly placed so that they are easily accessible for rapid use. Although the highly visible colors and enclosure design of existing AEDs have great value for public access, such as in airports and shopping malls, these bright colors of existing AEDs become a very negative aesthetic aspect of existing AEDs for home and office use. This negative visual aesthetic aspect of existing AEDs will often prevent the AED from being placed in an ideal location in the home or workplace such as a central location where it can be quickly and easily accessed in the event of an SCA, and not hidden away in a closet or drawer because it is obvious and ugly. Just as in the case of internal-integral batteries and their great utility when portability is needed (despite their well known reliability and cost problems), high visibility is also a very desirable feature for public access AED placements, but is highly undesirable in other AED placements such as in homes and offices where it is desired that the personal AED be placed aesthetically and centrally so as to be at the ready for rapid access and immediate use when needed.

By designing my AED to have no requirement to test or replace batteries, and by designing to provide the user the option of various selectable, aesthetic outside decorations, coverings, or indicia, my new AED is ideally suited for aesthetic placement and personal use in the home and workplace, locations where low cost, pleasing esthetics, and prolonged, maintenance free shelf life are all essential and are satisfied with my new design.

New AED Business Model Invented for Broad Deployment of Personal AEDs for Private Access

The traditional AED business model is a traditional sales and marketing product business model or method. This old AED business model currently provides the mechanism for sales of all existing AEDs by the various companies manufacturing and distributing them, and the purchase and placement of AEDs by the various agencies purchasing them. This traditional AED business method or model comprises the advertising, promotion, and sales of new AEDs designed for multiple patient use and refurbishment in the field by the user after use or expiration, and these AEDs are sold to both agency and individual customers. It is totally a product business model. In context of this AED patent application, “refurbishment” of AEDs in the field by the user is understood to mean the replacement of, after use of the AED or expiration of shelf life items, all consumable items such as electrodes and batteries and the subsequent testing and verification of proper functioning after such replacement of these consumable components. In the current product business model these duties are performed by the actual user or other agency personnel without return of the AED to the manufacturer for refurbishment. The owner owns an AED suitable for multiple uses with user refurbishment required every two years or after each use which ever comes first; my new business model comprises an AED placement service for or one use or for 4 more years shelf life, which ever comes first. After either of these events, the AED can be discarded entirely or the service can be renewed if desired for another single use or period of shelf life. The cost of the renewal service is paid for by the customer, the renewal service cost being less than one half of the original cost of the service, where the original service included a new, single use AED designed specifically for factory refurbishment after a single use or after shelf life expiration.

As previously described, all existing AEDs are designed for multiple patient uses with user refurbishment in the field after use or shelf life expiration and without any required factory refurbishment. These existing AEDs are thus all equipped with user replaceable batteries (one to 10 of them) and patient electrodes (two of them), such refurbishment items being purchased by the user from the manufacture or a distributor of such refurbishment items for AEDs. This multiple patient use, with user refurbishment of the AED in the field before next use, is advantageous and appropriate in many circumstances, since many public access AEDs and EMS managed AEDs are in fact expected to be used repeatedly, perhaps even several times in a single day. Further, since the users of such high use devices are often EMS professionals and as such, they are likely well trained, trained not only in the use of these AEDs to save lives, but also in their proper refurbishment and functional validation after use.

Generally, the design of current AEDs for multiple uses and user refurbishment in the field is appropriate for locations and missions where it is reasonably anticipated that the device may be used several times in a week, or at least many times in a year. But, for AED placements in private homes and workplaces where the AED will be used very rarely, if ever, and particularly where such ownership and very infrequent use is anticipated to be by non-professionals, the design of all current AEDs is inappropriate and inadequate in many aspects:

1) high acquisition cost for a device that may never be used even once,

2) required periodic testing by the owner, plus the maintenance and replacement of batteries and electrodes every two years to assure reliability even when the AED is not actually used,

3) short shelf life due to exclusive power by internal-integral batteries,

4) visually and aesthetically not suited for placement in a central location since existing AEDs are visually very obvious, and considered ugly visually in a home environment, and,

5) mandatory AED refurbishment by the user after an actual use or shelf life expiration.

The Inventive New AED Business Model is an AED Placement and Refurbishment Service . . .

It is the purpose of this invention to provide a new AED business model which provides a service to provide a personal AED designed for private access and single patient use. Further, this business model requires and provides a mechanism of factory refurbishment when needed, and which provides a low cost AED of greater reliability by requiring no maintenance during its 4 year or greater shelf life. The inventive AED business method described herein, is to market an AED for Private access via a service that delivers a single-use personal AED to the customer specifically for use in the home and workplace at substantially lower costs than existing public access devices, which are all designed and sold to be user refurbishable. These business model goals can be best achieved by an AED design which totally eliminates the use of internal-integral batteries as the primary or only power source, since such batteries have been the major weak link in the reliability and unattended shelf life of all existing AEDs. This lower cost per use AED is achievable because my business model and AED are designed for single patient use, to use no user replaceable batteries for AED power, and to be refurbishable after use only at the factory or other authorized refurbishment center.

Clearly, the existing AED business model, integrated with the existing AED design, is not designed for, and not conducive to, the widespread availability of AEDs in the average person's home or workplace. My invention is designed to correct these AED problems for home and workplace deployment.

New AED, Integrated with New Business Method, Enables Personal AEDs for Private Access

For “Private access” defibrillation using “Personal AEDs” , the problems with the existing AED business model, as well as the existing multiple-use AEDs are described above, and are severe. For instance, those persons that wish to have the security and comfort of knowing that an AED is no more than seconds away from them in their home or workplace must purchase an AED which is actually designed for multiple-uses and for user refurbishment in the field, both of which increase costs and complexity for the nonprofessional user.

For AED deployment into private homes, where it is very improbable that a personal AED will be used more than once in 4 years, (and in most cases the personal AED will never be needed and never used, just like most home fire extinguishers are never needed and never used), there is a severe mismatch between the needs of these users needing private access AEDs and the design, complexity of maintenance, and costs of existing AEDs. Such mismatch of needs is also reflected in the current AED business model which is purely a product sales business model. In contrast to the negatives enumerated above regarding current AEDs and the current AED business model, my new AED and service business model are designed to enable personal AEDs for private access by providing the individual user/owner:

1) AED with low acquisition cost for a single use only,

2) AED with no required periodic testing, maintenance, or replacement of batteries and electrodes,

3) AED with long shelf life of at least 4 years with no maintenance required during that period,

4) AED which is customizable by the user to provide a non-obvious as well as a visually and aesthetically pleasing presentation even when the AED placed in a visually obvious location, and

5) AED refurbishment by factory (or authorized refurbishment center) service after an actual use of the AED or its shelf life expiration.

This combination of AED and business model features and synergies realistically enables the placement of AEDs in most homes and workplaces at a cost of less than $0.50 per day, a cost substantially less than a security monitoring service, a cable TV service, or a connection to an internet service provider.

### SUMMARY OF OBJECTS AND ADVANTAGES

In view of the foregoing description of the disadvantages inherent in the known types of AEDs powered exclusively by integral batteries and their distribution by the existing business model of AED distribution as a product which is now present in the prior art, and having described many of the advantages of my inventive AED and new business model, several objects and advantages of the present Patent Application of Maynard Ramsey for “AUTOMATIC EXTERNAL DEFIBRILLATOR POWERED BY ALTERNATIVE AND OPTIONALLY MULTIPLE ELECTRICAL POWER SOURCES AND A NEW BUSINESS METHOD FOR SINGLE USE AED DISTRIBUTION AND REFURBISHMENT” are:

(a) to provide a highly reliable personal AED for home and office use which requires no internal-integral batteries and which is powered directly by electricity of 120/240 VAC (as is available in all homes and workplaces from wall outlets, etc.) or powered by external DC electrical power (as is found in all motor vehicles), hence overcoming the attendant shortcomings of the prior art AEDs which are all exclusively internal-integral battery powered;

(b) to provide an AED that is more reliable than the existing AEDs, which are all powered exclusively by internal-integral batteries, by optionally allowing external power to be used such as 120/240 VAC or external DC battery power such as from a motor vehicle battery, s well as internal-integral AED batteries;

(c) to provide an AED which is powered using 120/240 VAC from ordinary home electrical outlets or from AC of any source,

(d) to provide an AED power module, with or without built in power batteries, that designed such that it both physically and electrically replaces the traditional internal-integral battery of existing AEDs, and provides electrical connection to external power sources, such as 120/240 VAC or external DC battery power, and thus allows external power to be used in place of the regular internal-integral battery, thus providing the existing AEDs many of the reliability and shelf life advantages of my inventive AED

(e) to provide an AED that is designed specifically for single use and for factory only (or authorized service center) refurbishment;

(f) to provide an AED that requires no maintenance of any kind for its entire shelf life

(g) to provide an AED that has a greater than 4 year shelf life

(h) to provide an AED which has a long, non-tangling power cord that will allow the AED to be quickly connected to a source of external power, such as an external source of DC power or external source of AC power, and still reach the cardiac arrest victim with the AED unit, even when outside or in large size rooms in homes and workplaces;

(i) to provide a multi patient use embodiment of my new 120/240 VAC or external DC powered AED that is designed with a long automatically retractable power cord so that the AED becomes easily user refurbishable in the field after use for those special applications where user refurbishability after use is considered essential;

(j) to provide an AED with substantially shorter, and more consistent, energy storage times (so that the defibrillation shock energy can be delivered more quickly, and if needed repetitively), than is possible by the existing AEDs powered exclusively by an internal-integral battery power source;

(k) to provide an AED which can rapidly and repeatedly deliver therapeutic shocks without the increased charging time caused by a partially depleted integral battery as is the case with all AEDs currently available;

(l) to provide an AED which can be connected to an external AC or DC source of power which either of which is capable of exclusively powering said AED and also capable of recharging an integral rechargeable battery if such a rechargeable battery is present;

(m) to provide an AED which can optionally be powered by internal-integral batteries or alternatively connected to an external source of power which is capable of exclusively powering said AED regardless of the state of the integral batteries;

(n) to provide an AED which can be simultaneously connected to both internal and external power sources and utilize one such power source preferentially, so that for instance, if external power is present, the AED would utilize that external power in preference to its internal-integral batteries, thus conserving said internal-integral batteries for use when an external source of power is not available, and also reducing multiple shock recharge times;

(o) to provide an AED which can be powered by an integral small electrical generator, wherein the generator is human powered;

(p) to provide an AED which is powered using 120/240 VAC power where said power is created by using a DC to 120/240 VAC power inverter energized by a DC power source supplied by at least one of an electrical generator, a mobile battery pack, or other external battery, such as found in any motor vehicle, watercraft, or aircraft;

(q) to provide a four year or more maintenance free AED that is manufacturable at lower cost than the existing integral battery powered devices thus creating a true “Personal AED”;

(r) to provide an AED that is more reliable, lighter in weight, and more affordable than existing integral battery powered devices due to the simplicity of design and construction afforded by powering the new AED using an external power source such as an external DC battery, generator, or 120/240 VAC current instead of integral DC batteries;

(s) to provide an AED with an enclosure that is designed to be optionally decorated, mounted, and displayed so as to be as visible as a bright red, orange, or yellow fire extinguisher would be, or alternatively and selectably by the owner, the new AED can be decorated, mounted, and displayed as a book or photograph would be when placed on a shelf or table, and thus the AED can be aesthetically integrated, in a non-obvious way, into the pleasing decor and environment of homes and workplaces, but still the AED is still close at hand for instant use if needed;

(t) to provide an AED that is factory sealed, (optionally after filling the AED with moisture free gas such as nitrogen), such sealing designed to prolong shelf life, discourage and/or reveal tampering, and to prevent environmental contamination of the AED by excluding all water, dirt, dust, and other contaminants, but sealed such that the AED is easy to open and use when needed to rescue an SCA patient;

(u) to provide an AED that is more effective at defibrillation of difficult SCA patients than currently available AEDs, (which use only two surface electrodes and only single biphasic shocks), by providing means for an optional defibrillation mode of rapidly repetitive shocks;

(v) to provide an AED that is more effective at defibrillation of difficult SCA patients than currently available AEDs, (which use only two surface electrodes and only single biphasic shocks), by a design that optionally provides for utilizing additional body surface electrodes beyond the two used by existing AEDs, and further provides for optionally using alternative electrodes, such as esophageal and cardiac electrodes, that are closer to the heart and more effective than the traditional surface defibrillation electrodes;

(w) to provide an AED that optionally provides up to a 30 second delay in shock delivery after establishing the presence of ventricular fibrillation, such delay being used by the AED to optimize shock timing by analyzing the ECG pattern of ventricular fibrillation and delaying the defibrillation shock until such ECG analysis suggests that the heart is most likely to be defibrillated, such analysis being made on the basis of frequency, amplitude, and or morphology of the ECG waveform;

(x) to provide an AED that is powered by 120/240 VAC or external DC power, but said AED also provides an internal-integral battery for powering a time keeping and shelf life expiration annunciation function which is active while the AED is being stored, but so designed that the AED is fully functional when connected to an external power source even if the shelf life timing battery fails;

(y) to provide an AED that is powered by 120/240 VAC or external DC power, but said AED also provides an internal-integral battery capable of powering the AED as well as the time keeping and shelf life expiration annunciation function which is active while the AED is being stored, but so designed that the AED is fully functional when connected to an external power source even if the internal-integral AED battery fails to power the unit during an attempted rescue of an SCA victim, thus providing a backup power source in case of failure of the internal-integral battery;

(z) to provide a service oriented AED business model or method whereby single use AED access is sold or rented to the user by providing them a low cost AED designed for “single use with factory refurbishment” (SUFR), and thus greatly increasing the reliability of the AED and reducing the customer's cost per day of AED access since refurbishing the AED after use or shelf life expiration is less costly than simply discarding the single use AED after one use or after expiration of its shelf life.

Other objects and advantages of the present AED and the new AED access service business method will become obvious to the reader and it is intended that these objects and advantages are within the scope of the present invention.

To the accomplishment of the above and related objects, this invention may be embodied in the form illustrated in the accompanying drawings and text, attention being called to the fact, however, that the drawings are illustrative only of certain features of certain embodiments and that the functions described and shown therein are, in many cases, achievable by alternative methods from those indicated for schematic and simplicity purposes. Further it is to be understood that some aspects of my invention are not specifically illustrated in the drawings, but that all aspects of my invention are fully described in the text such that one of ordinary skill in the art could, using such descriptions, practice my invention based on the written disclosure alone or in combination with the drawings when appropriate.

### SUMMARY OF THE INVENTION

The present invention provides a new, “no internal-integral battery required for proper functioning” AED for home and office use powered by 120/240 VAC as exists in ordinary mains electrical power available in most homes and workplaces or by external DC power such as found in motor vehicles and the like, even if internal-integral batteries are optionally present. The highly reliable AC power source is 100-120 volts alternating current (VAC) in North America and Japan and 210-250 VAC in much of the rest of the world. Regardless of whether it is 120 VAC or 240 VAC, this mains power can be utilized for providing the energy for a highly reliable AED instead of internal-integral power batteries as is done in all other AEDs. The construction of an 120 VAC or 240 VAC powered AED presents additional safety considerations and requirements as compared to the low voltage DC battery powered devices. These additional safety considerations must be dealt with in the design of the AED which either exclusively or optionally uses 120/240 VAC, but the increased reliability of this ubiquitous power source, the expanded shelf life, and the reduced costs provided by using ordinary line power, rather than exclusively internal-integral battery power as all current AEDs use, creates both obvious as well as unexpected benefits. These great benefits more than compensate for the additional design constraints implied by powering my AED by 120/240 VAC, particularly when the AED is acquired for personal, single use, in the home or work place. Many similar benefits are achieved when my AED is powered by external DC power when available as compared to internal-integral only DC battery power.

Similarly, by designing a new AED to utilize 120/240 VAC or external DC power as its only energy requirement for proper functioning (whether or not internal-integral batteries are optionally present), the reduction in AED cost, the increase in AED reliability and utility, and the increased AED shelf life enable the development of a totally new business model for my AED's distribution. This new method is described as the “Single Use with Factory Refurbishment” (SUFR) AED access service business model or method. This new SUFR AED business method will permit the low cost acquisition of a new 120/240 VAC and/or external DC powered AED for approximately 10-20% of the average initial cost of an existing internal-integral DC battery powered, user refurbishable AED. Further, since in at least one embodiment there are no internal-integral batteries to check or test in my new external DC or 120/240 VAC powered AED, and consequently no batteries to replace periodically, additional cost is saved, and reliability greatly enhanced. Likewise, since there is no need to open or operate the device for testing of batteries, my external DC or external 120/240 VAC powered AED can be filled with a dry gas such as air, nitrogen, or argon, and hermetically sealed at the factory to prevent intrusion of dirt, dust, and moisture during storage. Another benefit of this factory sealing is that it may prevent casual tampering with the AED, but at the very least, such factory sealing and date and shelf life expiration stamping make tampering, prior use, or expiration of the shelf life obvious to the observer. This sealing thus insures proper AED functioning when the AED is needed to treat an SCA and thus it eliminates the tragedy of an AED attached to the SCA patient in need of defibrillation and then finding that the batteries are dead or too weak to function properly or the electrodes are dry and will not adhere to the patients chest. This tragedy will usually result in the death of the SCA victim, who would probably have been saved otherwise.

In my inventive AED and service business model, after a single patient use, or the expiration of the 5 year or greater shelf life, the AED is returned to the manufacturer or authorized refurbishment center for complete refurbishment. A complete refurbishment consists of replacement of the single use items which are present, such as the internal-integral batteries, the power cord, and the patient electrodes, as well as performing any software or hardware upgrades which may be non-mandatory, but which nonetheless are desirable to be performed on the AED returned for refurbishment and before return to the owner. The AED is tested, verified as performing to specifications and then is optionally sealed and date stamped with the shelf life expiration indicated. Unless otherwise specified, the term refurbishment or complete refurbishment is understood to include all of the replacement and performance test and packaging and sealing items as are applicable to the AED being thus refurbished.

Continuing the description of my new SUFR AED access service business model, as a part of this new business model which provides users with 4 or more years of immediate access to a maintenance free AED, a refurbished and fully tested AED is again factory sealed and then redeployed either to the same customer who returned it originally, or to a different subscribing customer. This substitution of AEDs after refurbishment is possible, and even desirable, because all AED units of a specific design type are identical in look, feel, function, and effectiveness. Under this new SUFR AED business method, the cost of the refurbishment service is less than one half the initial cost of AED service, further reducing the overall “cost per use” or “cost per year” of access to this new AED. Also, since all AEDs of a given design series function identically as described above, the business model and method includes the several optional methods of refurbishment service. One is where the owner returns a used or expired AED and awaits its refurbishment and return. In another method of refurbishment service, the owner requests shipment of a replacement AED (which has been previously refurbished), and then returns their original used or expired AED to the refurbishment center in the same shipping carton.

This latter method is particularly appealing to a subscriber who does not want to be without AED access while their original AED is sent to be refurbished. However, even with the regular AED return and refurbishment service, the user is without their personal AED for less than 24-48 hrs after the use of their original AED on a patient. In the case of shelf life expiration, the pre-shipment service provides a refurbished AED so that the subscriber is never without the protection of access to a personal AED. The cost for this factory refurbishment subscription service (by either the factory or authorized refurbishment center), and any required upgrades to the AED's specifications, is less than half of the original new cost of my new low cost AED. Further, such factory refurbishment assures (in contrast to user refurbished AEDs as is practiced with all existing AEDs and the current AED business model) that the refurbished, factory tested, and sealed AED is returned to the owner in a condition that guarantees its reliable functioning over the restarted shelf life of 4 or more years. When an AED is over 10-12 years it is retired from service regardless of condition, and retired earlier if testing indicates such early retirement is appropriate. The major safety advantage of my SUFR AED access service business method is that each AED so refurbished is like new and hence unlikely to malfunction in its next single use as long as it has not exceeded it shelf life.

The business model described above is applicable specifically to the inventive AED which is designed specifically for said business model, namely, SUFR. Additionally however, the SUFR business model is also very applicable, and in many cases very beneficial, to all existing AED designs which are all designed for multiple patient use with user refurbishment in the field. The battery replacement power module here disclosed is also available as a part of the AED service model and hence allows existing AEDs, which are all internal-integral battery powered, to be externally powered and hence gain the reliability of my inventive AED with respect to usability with alternative power sources. This broad utility of our new SUFR AED business method is thus extended to all existing AEDs and is likely to be highly desirable to some segments of the existing AED industry as a whole, since in many cases the refurbishment of the current AEDs, after use on a patient or shelf life expiration, may not be readily accomplished by the owner or authority responsible for the safe functioning of the AED. Additionally, the replacement of current AED's internal-integral battery with the new external power enabling power module, prior to redeployment after refurbishment, will prevent many AED failures due to exclusive powering by internal-integral batteries when said internal-integral batteries fail. The option of alternative external AC or DC power sources (in addition to the optional batteries contained within the battery substitution power module) can maintain and support proper AED function when these optional batteries within the battery substitution power module are dead or inoperative for any reason. This substitute power module is for use with current designs of AEDs, but it is to be understood that in one embodiment of my AED where traditional internal-integral batteries are present, there are additionally present externally accessible connectors to permit connection of external AC or DC power, using an appropriate power cord, for either primary use or for backup use if the internal-integral batteries fail for any reason. Such ability to connect my AED to external sources of power enables the proper function of the AED regardless of the presence or state of internal-integral batteries and enables saving the SCA patient as a result.

Therefore, my new AED access service business method service, SUFR, includes the refurbishment, the optional addition of external powering capability to existing AEDs, and the testing of all existing and future AEDs. Also, the immediate shipment of a factory refurbished “foreign” AED, in exchange for a like “foreign” AED (“foreign” AED meaning a conventional AED, that is, an AED not originally designed for single use and/or external power) unit in need of refurbishment and testing after patient use, would also be available for AED access service subscribers with existing, but non-inventive, “foreign” AEDs. Thus our SUFR AED access service business model captures the refurbishment of not just the inventive AED and its derivatives, but also all of the existing AEDs after use on a single patient or shelf life expiration. It also provides the option to extend the utility and reliability of the option of external powering to all existing AEDs by replacing the internal-integral battery module with my new external power access module which also optionally contains internal batteries as well. The applicability of the new business method to all AEDs greatly enhances the utility and profitability of the new SUFR business method service. Likewise, since SUFR introduces a major change in the refurbishment and testing of existing AEDs, previously all done by the user or owner of the AED in the field without factory supervision, SUFR can greatly enhance the reliability of all AEDs refurbished at the refurbishment center, thus increasing the reliability of the whole “public access” AED industry, in addition to truly creating the new “private access”, personal AED access service industry.

### BRIEF DESCRIPTION OF THE DRAWINGS

In the drawings, closely related figures have the same number, but different alphabetic suffixes.

FIG. 1 shows a perspective view of an automatic external defibrillator (AED) which implements the current invention of being optionally powered by 120/240 VAC or external DC power, and equipped with a long coiled, non-tangling power cord for plugging into an ordinary wall power outlet or to a source of DC power such as a motor vehicle.

FIGS. 2a-2b shows the coiled power cord (AC plug shown attached to power cord and with DC power adapter) before packing and after packing in a power cord container for non-tangling, rapid, and easy deployment when needed

FIG. 3 shows a general schematic diagram of the electrical control system of my AED which in this embodiment is powered by 120/240 VAC exclusively whether from the wall or from a DC source connected to a DC to AC inverter

FIG. 4 shows a method of creating the high voltage energy from a 120/240 VAC power source

FIG. 5 shows another method of creating the high voltage energy from a 120/240 VAC power source

FIG. 6 shows another method of creating the high voltage energy from a 120/240 VAC power source

FIG. 7 shows schematically an AED embodiment that uses optionally either AC power, internal-integral DC battery power, or external DC power from any source

FIGS. 8a-8d shows schematically primarily the powering considerations of an AED embodiment that uses one or more external power sources as alternatives to optional internal-integral batteries, such that, even if said internal-integral batteries are present, the external power sources are connected to the AED by a power cable, said cable being suitable for AC or DC power

FIGS. 9a-9c shows three alternative patient electrode configurations utilizable by the AED using combinations of body surface, esophageal, and cardiac electrodes

FIGS. 10a-10d shows four different presentations of the AED when being stored for ready access in the home or office, and in FIG. 10e the AED sealed within a protective cover with quick open pull tab

FIG. 11a shows the optional external power module (with affixed power cord) for existing AEDs which permits use of AC or DC external power (and optional internal-integral batteries within the power module) whereby this power module replaces the standard internal-integral or integral battery pack on all existing AEDs

FIG. 11b shows the optional external power module without the affixed power cord and with connectors for connecting external sources of AC or DC power

FIG. 12 shows a flow chart of the new “single use with factory refurbishment” (SUFR) AED business method for AED distribution and AED refurbishment for both the inventive single AED and multi-use existing AEDs

FIG. 13 shows a summary comparison of the new AED and SUFR AED access service business model versus the old style AED and AED product sales business model

### DETAILED DESCRIPTION OF THE PREFFERED EMBODIMENTS

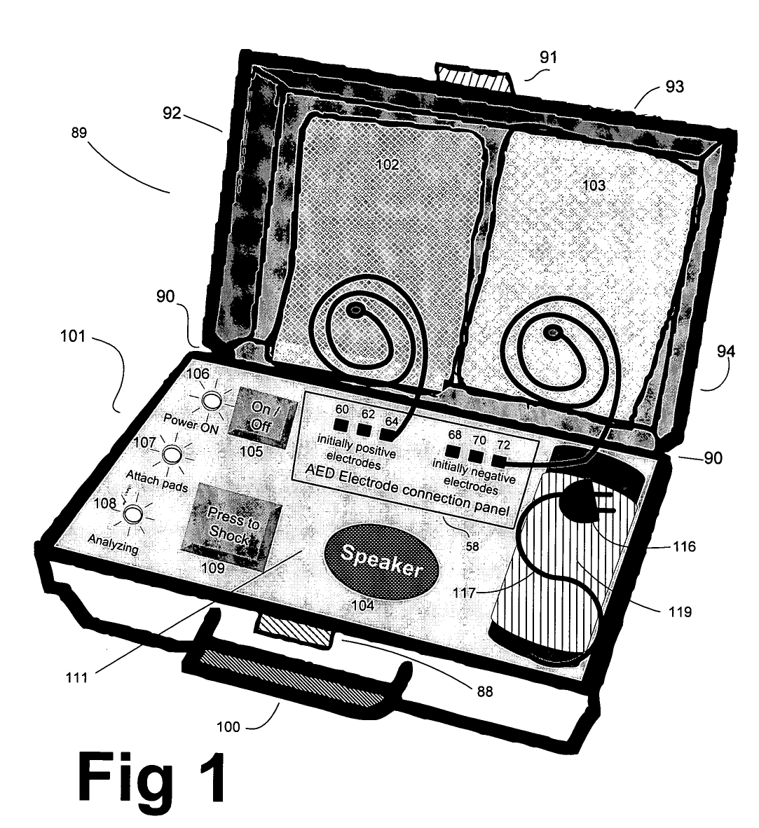

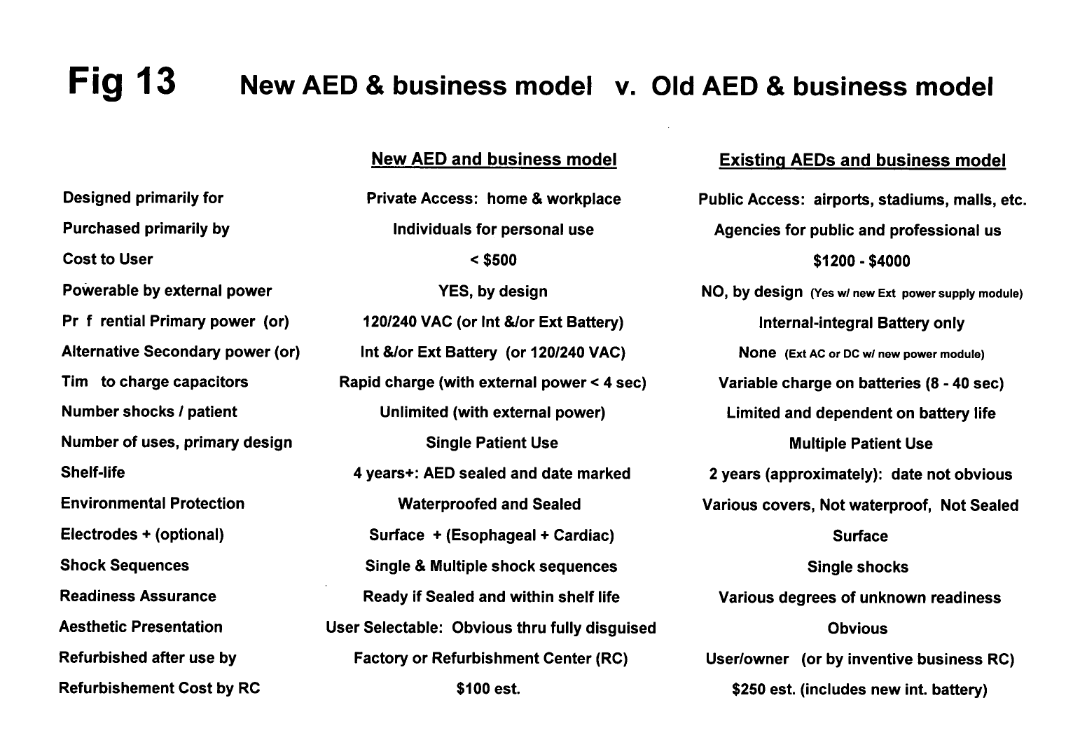

Turning now descriptively to the drawings, FIGS. 1-13, in which similar reference characters denote similar elements throughout the several views and schematics. FIG. 13 summarizes the new inventive AED and its new business method with the existing AEDs and their business method and is instructive to peruse as a means of overview of the bulk of the invention.

The attached figures illustrate a new AED for home and office use, said AED being powered by 120/240 VAC, which is most typically supplied by the local power utility company or powered by external DC power, most typically the 12 volts DC available in most motor power vehicles and water craft. Optionally one or both of these power sources are available as well as internal-integral batteries as are the sole source of power of all existing AEDs. The method of utilization of AEDs by trained or by untrained persons is well know in the art and is virtually identical amongst all of the existing prior art AEDs and hence will not be described further except where aspects of my invention require such description. The descriptions below are therefore devoted to the specific elements, and their functions, as they relate to my inventive AED coupled with its new business method.

In all of the figures, the ECG signal path, when present, is represented by the pair of dashed lines circled and labeled as 175. The wires which connect the AED to the patient electrodes are circled and labeled as 141 and supply ECG signal to the AED for ECG analysis for a shockable rhythm, and upon the detection by the AED of a shockable rhythm, said wires deliver to the patient the high voltage, high energy shock designed to defibrillate the patient.

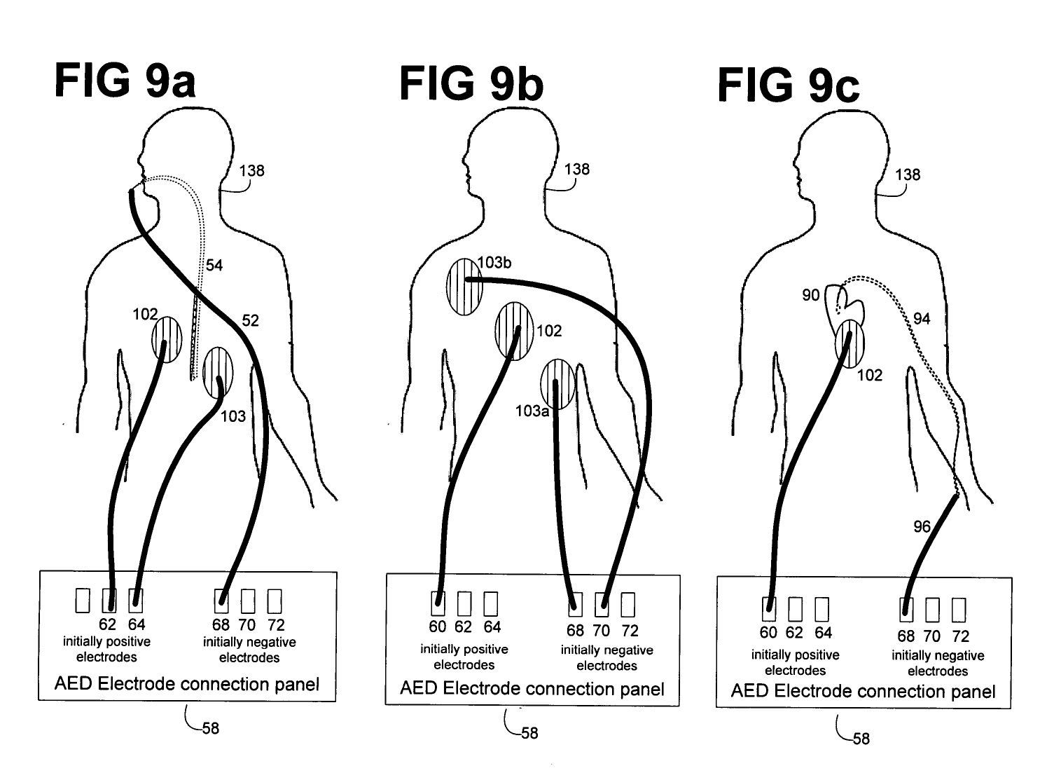

Similarly, the body surface electrodes which are to be attached to the patient in the various figures are labeled 102 and 103 and are visually represented as the self sticking type of surface electrodes well know in the art. However, it should be understood from the start of these descriptions, that my inventive AED is designed also to optionally utilize other forms of electrodes which are not now used by current AEDs, but which when used with my AED may make the difference between defibrillation success and failure. Namely, in most embodiments, one or more of the surface electrodes 102 and 103 illustrated in the figures may be optionally replaced or augmented by either one of, or both of, additional surface electrodes, esophageal, and/or cardiac electrodes as more fully described later and specifically depicted in FIGS. 9a-9c. It is however now to be noted that both cardiac electrodes and esophageal electrodes are naturally positioned substantially closer to the heart than are the body surface electrodes illustrated in the figures as 102 and 103 and hence such alternative electrodes are generally more efficient in the delivery of energy to the heart and hence may be successful at defibrillation when surface electrodes alone are not successful. In this way, my AED, when used with these alternative and/or additional surface and non-surface electrodes may save a patients life that otherwise would be lost if only the traditional two surface electrodes were used. The attachment means of these alternative electrodes to the AED is similar to that attachment means utilized by the body surface electrodes and said attachment means optionally permits individual substitution of one or more of the surface electrodes by one or more of the cardiac or esophageal electrodes at the option of the user. These optional electrode configurations will typically be utilized by professional EMS personnel when using my new AED, but are optionally available to the home user. In other embodiments, additional electrode connectors are provided on the AED so that the user can attach additional electrodes beyond the traditional two 102 and 103, so that the shock is delivered through three or more electrodes in any combination of surface, cardiac, or esophageal electrodes, three examples of which are illustrated in FIGS. 9a-9c.

Referring now specifically to FIG. 1, which is an external perspective view of said AED 101, which comprises the enclosure case or housing of the AED optionally with a handle 100, and a cover or lid that may also be used for internal storage of the patient surface electrodes 102, 103, (or additionally optional esophageal 54 or cardiac electrodes 94 in illustrated in FIG. 9), prior to use on the patient. Said surface electrodes 102, 103 are optionally plugged into the electrode connector panel at time of manufacture and enclosure sealing in order to eliminate the requirement for the user to plug the electrode connectors of 102 and 103 into the connector panel 58 prior to use, thus saving one step in the defibrillation process.

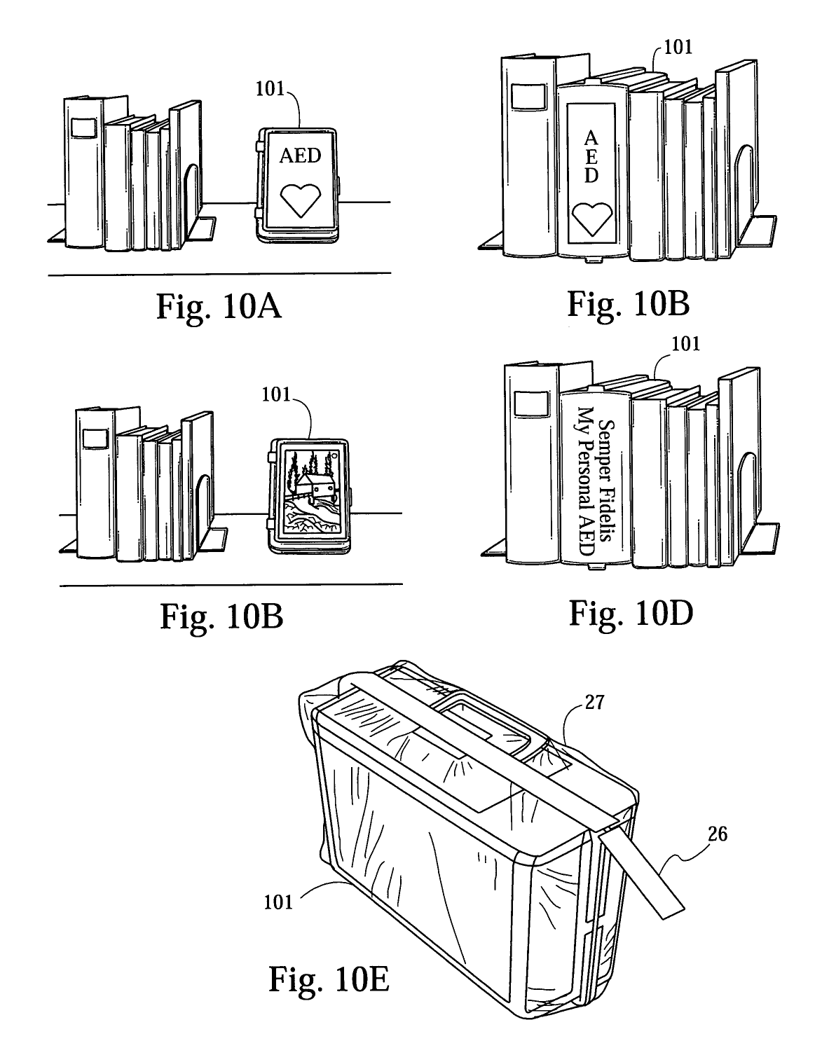

The AED enclosure is designed to contain all of the circuitry, cables, electrodes, and power connection devices (and optionally internal-integral batteries) so that when the enclosure is closed and sealed all components are securely contained within said enclosure. Since there are, in many of the embodiments of my inventive AED, no internal-integral DC batteries that need to be checked or changed while the device is in storage awaiting possible future use, the unit can be sealed so that moisture cannot enter and so that any previous use or tampering with the device will be obvious to those responsible for its storage, display, and eventual safe and effective deployment. Likewise, since external power is available, at least as an option, in all of the embodiments of my AED, even in those embodiments with internal-integral batteries, the AED can be completely sealed, since if the internal-integral batteries failed, as is sometimes the case, the AED could still be made functional by plugging it into an external source of AC or DC power. Though not shown in FIG. 1, but as shown in FIG. 10e as 27, the entire AED, including its electrodes, any optional internal-integral batteries, and the power cord for accessing external power, can be optionally completely sealed in a protective over wrap or package of paper, metal, or plastic or other such protective material to further protect it from water, dirt, dust or other contaminants, and to prevent, or make obvious, tampering prior to use. Said over wrap 27 being illustrated in FIG. 10e as a clear plastic over wrap. Also in accordance with my invention, such AED over wrap package has means for rapid removal such as with a quick opening tab as shown in FIG. 10e 26 such that this protective packaging does not impede rapid utilization of the AED when needed. Similarly, when such over wrap is utilized, it is also provided with means for tastefully mounting external decoration such as indicia, text, or photographs which are designed to obviously proclaim, or to subtly disguise, that the item behind such prominent or subtle facade is, in fact, an AED 101, various of such decorations are illustrated in FIG. 10.

Referring still to FIG. 1, the control panel 111 of the AED 101 is shown with various status indicators and annunciators such as a speaker 104, and optionally, the user controls for the AED, such as an ON/OFF switch 105. The control panel 111 provides user access to the various control functions which are optionally present and to the optionally present status indicators 106, 107, and 108 which indicate for the user where in the process of defibrillation they are at any given moment. The control panel 111 also covers the AED circuitry and any internal-integral batteries if present which are all safely enclosed below the front panel 111 and out of view and reach of the user. (In another embodiment, the internal-integral batteries would be accusable by the user.) Said status indicators and annunciators 106, 107, and 108 may vary in the various embodiments of the AED, and in some embodiments, one or more of them are optionally not present. For illustration purposes however, an ON indicator light 106 would indicate that the device is either plugged into a valid external power source or using internal-integral batteries and has been switched to the ON position using switch 105. If when switched to ON, if the ON is not lit, it would indicate that no power was reaching the AED and alternative power should be selected from external AC or DC power sources using the no-tangle power cord 119 or other available power cord for connecting to AC or DC external power. Other optional status indicators and user prompt indicators are the “attach pads to patient” indicator 107 and a status indicator to indicate that the AED is in the process of monitoring and analyzing the patient's ECG to determine if there is a shockable rhythm 108.

Switch 109 is pressed by the user to deliver the shock energy to the patient if the analysis determines the patient has a shockable rhythm, unless the AED is in the totally automatic mode, in which case the shock is delivered without a button press by the user. In some AED embodiments, the shock prompt is given to the user audibly or visibly and in other embodiments, the user is alerted that the AED is going to automatically deliver a shock to the patient and to stand clear of the patient.

In one embodiment of my AED, there is a unique and radical departure from all existing AEDs, in that in this embodiment of my AED there is no ON/OFF switch 106 and the AED is functional as soon as the user plugs the AED into the wall to supply 120/240 VAC electrical power to the unit or plugged into an external source of DC power. In this embodiment, the AED is disabled only when unplugged from the power source. The absence of an ON/OFF switch provides advantages, since by applying external power to the AED, the device is automatically powered to ON, and it is not required for the user to remember to switch the AED to ON and thus eliminates one more step that is required of the rescuer to perform before saving the life of the SCA victim. The manufacture of the AED with surface electrodes 102 and 103 already plugged into the appropriate connectors on the electrode connection panel 58 also saves the user precious time and eliminates yet another step in their necessarily rapid quest to save a life. Since initial rescuers using my AED are most likely not professionals, these simplifications of operating procedures could make the difference between success and failure.

Similarly, some embodiments will not have all of the status indicators and user controls that are illustrated in FIG. 1. In one embodiment of my AED, there are no controls for the user to operate at all, all functions being achieved automatically, beginning as soon as the AED is plugged into the AC wall outlet or external source of DC power. In all but the simplest embodiments, the speaker 104 which is controlled by the circuitry of the AED, will instruct the rescuer to place the electrodes on the chest of the patient and then to call the closest emergency agency, which in the United States is by dialing ‘911’, while the AED analyzes the victims ECG and automatically delivers, or asks the user to deliver, the defibrillation shock. Similarly, once powered, in all but the simplest embodiments which contain no voice prompts, the speaker 104 will advise the rescuer that a shockable rhythm has been detected and to stand clear of the patient and if not in automatic shock mode, to press the “Press to Shock” button 109. In the fully automatic mode, it is anticipated that after placement of the electrodes on the victim, that the patient will be automatically and successfully defibrillated and breathing and talking again before the rescuer is finished calling 911. It is an object of my invention to make saving an SCA victim by an untrained person that easy and that sure.

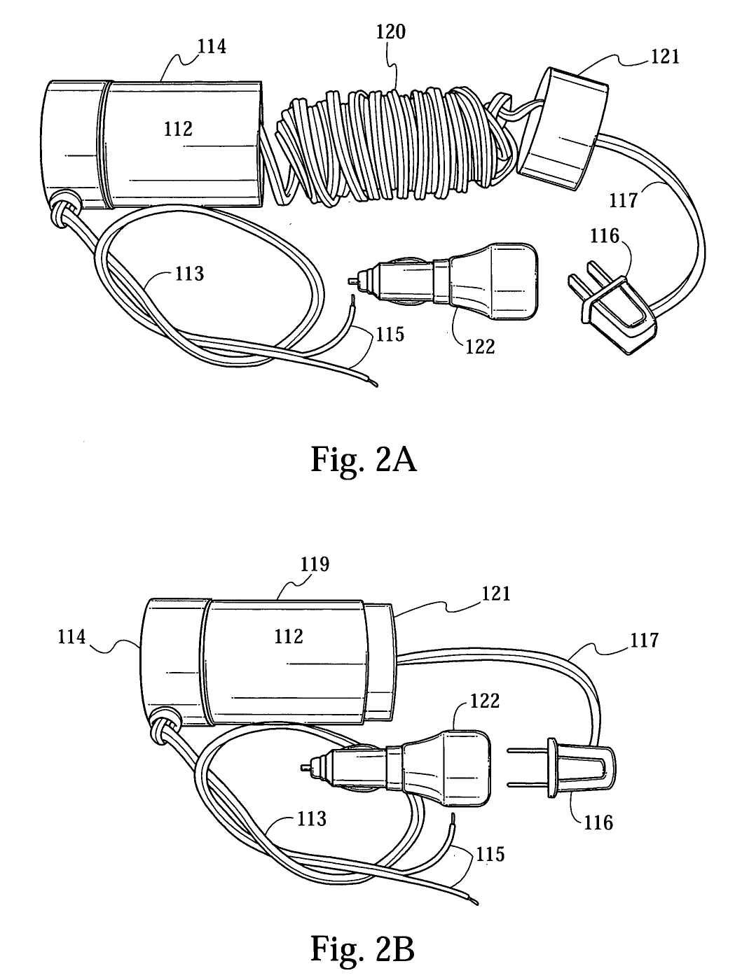

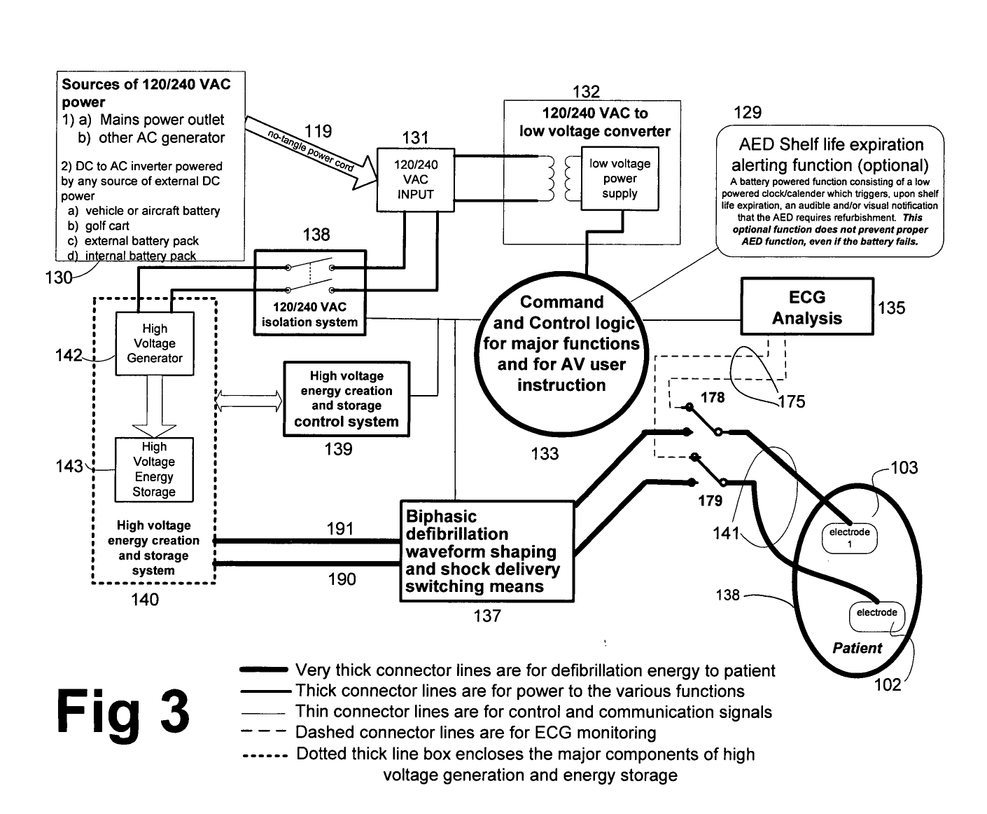

Continuing with the description of the inventive AED and referring specifically to the AC and/or DC external power cord system in system in FIG. 2 and the overall system diagram in FIG. 3. The diagram in FIG. 3 illustrates that the AED comprises various functions, many of which are well known in the prior art as a part of all AEDs, but which are included for clarity and completeness.

My AED, being powered by in most embodiments, (and at least optionally so if internal-integral batteries are present as they are in all existing AEDs), by external sources of AC or DC power, my AED requires a power cord to be plugged into a source of 120/240 VAC (which will be most commonly an ordinary wall 120/240 VAC power outlet) or external source of DC power (which will be most commonly an ordinary 12 volt DC cigarette lighter type power outlet in a motorized vehicle) requires a cable to connect the AED 101 to the source of external power, said source of external power being potentially some distance from the fallen SCA victim.

FIGS. 2a and 2b are illustrative of a design which will provide a long, no-tangle power cord for accessing external sources of power which is long enough to reach from a fallen SCA victim to the nearest AC or DC power outlet, said power cord and plug being packaged in such a way within or about the AED 101 so as to remain untangled both in storage as well as during the rapid deployment of said long power cord from the site of the fallen victim to the nearest source of applicable power. Alternatively, the power plug could be plugged into an external source of power and the ADE moved to the SCA victim while the no-tangle mechanism allows the power cord to pay out as the AED is moved from the power source to the SCA victim. Looking specifically at FIG. 2a, the long power cord 120 is shown coiled in a multiple layer cylindrical coil before it is inserted into the cord container means, shown as a cylindrical container 112. When the coil of wire 120 is inserted into the container 112 and end caps 114 and 121 attached, the great bulk of the entire power cord is safely stored within the completed power cord assembly 119 where it is both protected and stabilized during transport and during extended storage. External to the cord container is a length of cord 113 for connection to the power input circuitry of the AED at 115, typically using a connector or soldering the stripped ends of the power cord wire directly to the circuitry of the AED. At the distal end of the long power cord is the power plug 116, here shown as a standard 120 VAC two pronged power plug (although other types are optionally suitable as well depending on the anticipated sources of external power), which is connected to a source of AC power by plugging it into a mating power outlet connector, typically a wall power outlet found in most homes, but optionally any source of 120/240 VAC, in those AED embodiments designed to function on AC power. In the case where the AED is functional on external DC power, another such no-tangle power cord is optionally present, but the power plug on this additional no-tangle power cord is such that it can directly access external DC power, typically 12 volts DC from a vehicles power output, cigarette lighter socket, or via gator clips attached to an external battery. Alternatively, to prevent the requirement for two power cables such as 119 when the AED is designed to permit use of one of the external DC or AC power sources, a single no-tangle power cord assembly 119 with plug 116 is present for AC use, and additionally, a DC power adapter 122 to change the AC plug 116 into a DC plug 122 for a cigarette lighter type socket is also provided with the AED. In this way, a single power cord can function as an attachment to either an external DC or an external AC power source.

Clearly, the default plug 116 on the power cord could be instead a DC type plug and an equivalent DC to AC plug adapter could be supplied is desired. Both methods are equivalent and the selection of the type of default plug is advantageously made to be the type of connector plug which would plug directly into the power source connector jack most likely to be used for external defibrillation power. For instance, if the AED is designed primarily for home or workplace, the default power connector plug 116 is most advantageously that for 120/240 VAC. Conversely, if the AED is designed principally for use in or close to a motor vehicle, the DC connector cigarette lighter type plug is advantageously supplied as the default power connector and a DC to AC plug adapter supplied for optional use with AC power. Since internal-integral batteries are the single most problematic portion of the design and implementation of existing AEDs, my new externally powerable AED achieves a new and higher level of reliability by allowing this use of alternative power sources which are ubiquitous and which are inherently more reliable than the internal-integral batteries of existing AEDs.

Referring again to FIG. 2a, whether powering the AED by external DC or AC power, the power cord assembly 119 provides for a rapid and tangle free cord extension when the device is to be used, and it keeps the cord safe in during transport and storage awaiting use. Since my AED is designed principally for single patient use with factory refurbishment, the power cord assembly as shown is not designed for user repacking in the field and will be repacked or replaced at the time of factory refurbishment, assuring that it is always reliable, properly packed for no-tangle deployment, and never worn out. Other methods of power cord packaging are optionally useful in alternative designs and embodiments. One alternative method would be to place the coil of wire within a compartment of the AED itself where it would be safe and prevented from tangling. Another alternative method is to fold the cord in a rectangular form or into a Z-form within a suitable integral compartment of the AED or a separate cord container. Another approach is to wrap the coiled or folded power cord in a thin plastic wrap such as Saran or a light metal such as aluminum foil, said wrap containing the cord until deployment. Another method of tangle free cord extension when being used is an automatic retractable cord reel as is found in some home electrical appliances such as vacuum cleaners and the like. This alternative method of providing tangle free deployment of the power cord has an additional advantage of tangle free retraction of the power cord after use which eliminates power cord re-packing or replacement as a part of the refurbishment process. In this embodiment, the retractable cord would provide the user the ability to refurbish the AED power cord packing in the field since the tangle free cord re-packing would be made automatic and tangle-free by the retractable reel. This latter capability might be important for professional use of my AED when multiple use and field refurbishment were deemed necessary.

For all of these means of AED power cord storage and deployment, the essential requirement is that it be designed such that the power cord never tangles or kinks during deployment of the power cord by the user. Such a power cord tangling malfunction during deployment would possibly prevent the AED's patient electrodes 102 and 103 from reaching the victim when the AED was plugged into an external AC source or to an external source of DC power, due to the foreshortening of the power cord due to a tangling within the cord storage and deployment means.

Referring now to FIG. 3 which is a block diagram schematically illustrating the general design of an externally powered AED, the external power being in this figure external AC power. Beginning with the 120/240 VAC power source block 130 and the connecting no-tangle power cord 119 (previously described in FIGS. 2a-2b), it is seen that, in accordance with my invention of a “no internal-integral battery required” AED that all power input to the device as shown in this embodiment is from an external 120/240 VAC source. Most typically the ordinary household wall current will be the source of such AC power, though it may optionally be derived from alternative sources such as motor driven generators or DC to AC inverters as may be found in boats, motor vehicles, and aircraft. The no-tangle power cord assembly 119 provides the exclusive power input to the AED in this embodiment, said 120/240 VAC power being utilized within the AED for all required functions, principally:

1) to supply AC electrical power to the line power AC voltage to low voltage converter and low voltage DC power supply 132, said low voltage DC power being used to power the “Command and Control logic” function 133 and subsequently to control the analysis, charging and switching function required to deliver the shock to the patient when required, and,

2) to supply the 120/240 VAC power to the high voltage energy creation and storage functions 140. This high voltage energy creation and storage functional block 140, is in this embodiment supplied with raw AC line current through a suitable line voltage isolation system 138 to protect the patient from mains power in the event of a failure, and shown here as a mechanical relay activated by control means 133, but it is understood that semiconductors may be used to effect this isolation protection function as well. In FIG. 3 the line voltage isolation system 138 is shown in the ‘open’ or disconnected position as would be the case when energy was being delivered to the patient; the relay would be ‘closed’ during the time the AED was charging the storage capacitors using block 140.

Though functionally this line voltage isolation unit 138 is not required, it provides for greater safety in the event of failure of other AED components since the “Command and Control logic” function 133 could disable the high voltage creation circuitry by activating the line voltage isolation system 138 should it detect a malfunction of any component. Similarly, for further safety redundancy, one or more additional line voltage isolation systems such as shown 138 could be arranged in series for independent control by the control logic function 133. Such arrangement would assure that failure of one such line voltage isolation system would not result in unwanted and hazardous flow of current within the AED, and thus preventing it from potentially reaching the patient through electrodes 102 and 103. However, the reliability of modern electronic devices such as semiconductors and relays makes the possibility of unwanted current flow very remote. Though shown as relays for simplicity, other solid state devices are available which can electronically perform the same isolation function.

The “Command and Control logic” function 133, is the central logic which governs the overall function and processing for the AED, including analyzing the ECG 135, charging the storage units 143, shaping the waveform 137 when the stored energy is delivered to the patient through switches 178 and 179. As illustrated, there are numerous physical and logical connections to the various key functional blocks of the AED, namely, line voltage isolation system 138, the ECG analysis block 135, the high voltage energy creation and storage block 140, and the shock delivery and shaping block which actually connects the stored energy to the patient and creates the desired waveform. The ECG acquisition and analysis block 135, is well known in the prior art and consists of circuitry and logic, generally supplied by the central processor 133, to determine if the patient has a shockable rhythm, namely ventricular fibrillation or ventricular tachycardia, and will not be further described. However, it is on the basis of the existence of said shockable rhythm that the command and control block 133 will cause the delivery of the therapeutic shock. If there is no shockable rhythm, 133 will continue to monitor the analysis of the patients ECG, ready to deliver the shock if and when it is indicated as being required.

Also shown in FIG. 3 is the functional block 133 labeled “Biphasic defibrillation waveform shaping and shock delivery means” which, under the control of 133 will switch the stored energy to the patient and at a chosen point, reverse the polarity of the energy applied to the patient to create the biphasic defibrillation waveform, all of these functions being well known in the medical literature regarding the development of the first biphasic waveforms and AEDs, such reversing function typically comprising an H-bridge circuit, also well know in AEDs and motor reversing circuits. The energy for the second, or reversed polarity, phase of the defibrillation waveform can come from the same energy storage unit 143 which supplies the first phase of the biphasic defibrillation waveform or from a separate stored energy source which is independent from the first stored energy source. In the embodiment where the energy for both the first and the second phases are supplied by the same energy storage unit, an H-bridge circuit, well know in the electronics art for reversing electric motors and the like, is used to reverse the polarity of the electric energy applied to the patient and is what creates effectively the biphasic defibrillation waveform. This second phase of the biphasic defibrillation waveform, at a chosen point, is switched off or truncated by switching or truncation means 137 well know in the art, typically employing relays or semiconductor switches.

In the second method of creating the biphasic defibrillation waveform, where the reversed polarity second phase of energy delivered to the patient is from a second high voltage energy source (not illustrated in the figures but identical in construction to that shown) separate from the first high voltage energy storage unit 143, the high voltage energy delivery from the first energy source must first be truncated by a truncation means before the reversed polarity second phase energy is delivered to the patient. Similarly, the second phase of energy delivery must be terminated by a truncation means to prevent the long “tail” of low amperage current that is believed to cause re-fibrillation, and hence result in a failure to defibrillate. All of these switching functions are contained in the block 137 and are implemented by electromechanical or semiconductor switches well known in the electronics art. All existing AEDs use a single bank of storage capacitors for both phases of the biphasic defibrillation waveform, but in my AED, it is anticipated that it may be advantageous in certain embodiments to use two separate banks of storage capacitors, one for each phase of the traditional biphasic defibrillation waveform, and similarly additional banks of storage capacitors if tri, quadra, penta or other multi-phasic waveforms are used.

Also shown in FIG. 3 is a block 138 representing the patient and the patient's chest and showing the attached body surface electrodes 102 and 103 on the surface of the patient. These patient electrode pads are of the standard type, well known in the prior art, but in the present invention, they may be optionally not packaged in outer foil jackets as is currently done, since the entire AED unit can be hermetically sealed (FIG. 10e 27), since in at least one embodiment it has no internal-integral batteries to test or to replace. In the AED external embodiment shown in FIG. 1, the wires for electrodes 102 and 103 are already attached to the AED via electrode connectors, thus freeing the user from having to attach them to the connection panel 58 and further simplifying the process of attaching the electrodes to the patient and making the system ready for operation. This optional hermetic sealing of the entire AED, including said patient electrodes 102 and 103, prevents the drying out of the electrodes and thus assures their usability when used within the published shelf life of the AED. However, even though it creates and additional step for the rescuer who must remove the electrodes from their package when wrapped in typically a foil type package, the sealing of the patient electrodes specifically in an individual hermetic package will generally prolong their shelf life as compared to sealing the entire AED in an hermetic package and such individual packaging of the electrode pads is preferred when extreme shelf life is required.

For this description of the protective sealing of my AED, refer to FIG. 10e. The ability to seal the AED totally within an hermetic over-jacket 27, or alternatively to seal the AED enclosure lid edges 92, 93, and 94 in FIG. 1 to the bottom of the AED enclosure 101, is highly advantageous since it assures that the product has not been tampered with and has not been damaged by water or other environmental contaminants during its entire shelf life storage.

When utilized, this sealed (27 FIG. 10e) over-jacket packaging of my AED is fitted with an obvious pull tab (26 FIG. 10e) or other mechanism for quick and easy opening in the same way that the emergency personal flotation devices stored in aircraft are so packaged and are fitted with quick opening tabs. The creation of an AED 101 which can be totally sealed and protected until actual use, is a major advantage of my AED design over the prior art devices. Said sealable, waterproof, and tamper proof AED design is enabled by my novel AED design which utilizes external power sources such as common household AC power or common vehicular 12-24 volt DC power and hence by specific design in a preferred embodiment eliminates the requirement for all internal-integral AED batteries as is required in all existing AED designs. Since there are no internal-integral batteries to test or to replace, there is no need to ever break the edge seal or the over wrap seal and access the AED until it is needed for saving the life of a victim of SCA, and thus an unbroken seal assures an undisturbed and truly ready AED when needed.

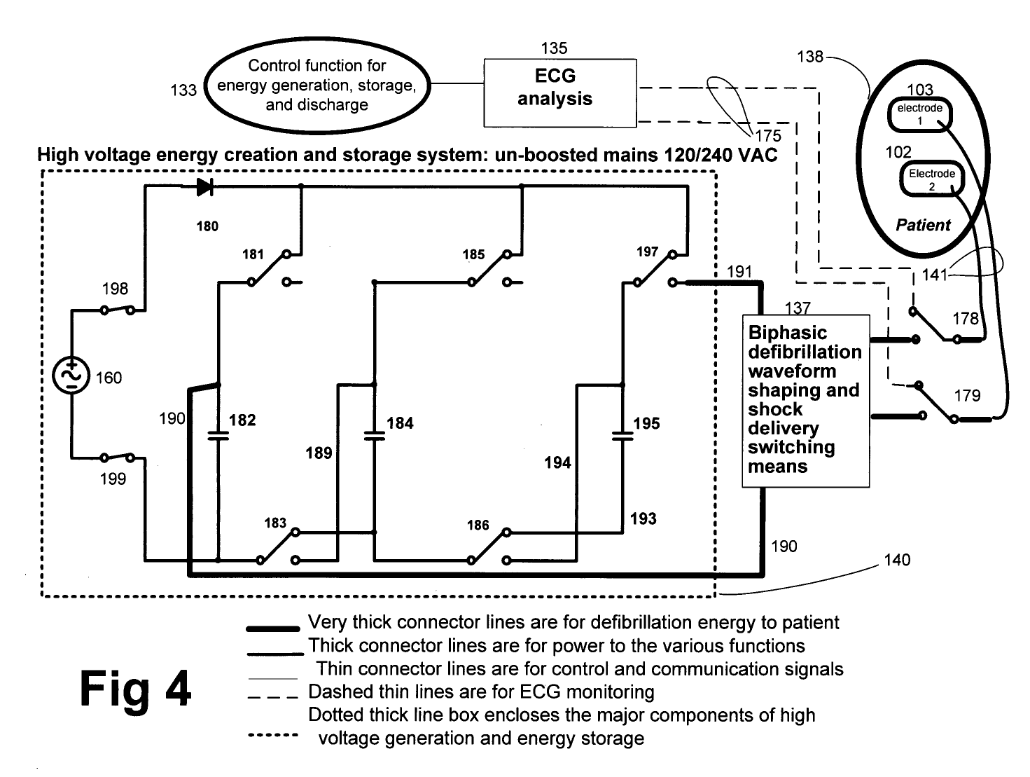

In the embodiment shown in FIG. 3, bock 140 is the high voltage energy generation and storage function circuitry, and is that portion of the AED which converts the electrical energy obtained from the 120/240 VAC power source into the high voltage energy which is stored in said energy storage means 143, and which can be discharged into the patient through electrodes 102 and 103, via the wave shaping and switching function in block 137, on command of the fibrillation detection and shock delivery logic of the command and control unit 133. In this embodiment, which utilizes exclusively the common household 120/240 VAC current to totally power the inventive AED, the presence of 120/240 VAC enables the creation of several different embodiments of block 140, the high voltage creation and storage block. Though three embodiments of this functional block are presented in FIGS. 4, 5, and 6, it is to be understood that there are many combinations of the elements shown, thus creating other configurations and designs, that accomplish the same ultimate function, that function being the creation high voltage, stored energy which is to be delivered to the patient via 137 under the control of 133 when there is determined by 135 to be a shockable rhythm.

Still referring to FIG. 3, block 129 describes a clock or timer function which is battery powered by a small internal battery which is not user testable of user replaceable and which is totally nonessential to the functioning of the AED. The purpose of this optional circuit represented by block 129 is to alert the owner when the shelf life of the AED has expired, or is about to expire, so that a replacement AED can be arranged before the expiring AED must be returned to the factory, or to the refurbishment center, for credit and/or refurbishment. Should this shelf life battery malfunction, such malfunction would in no way affect the proper functioning of the AED when connected to the required external power.

Looking now at FIG. 4, the first of three illustrated means for high voltage creation and energy storage, the figure is labeled as “un-boosted mains 120/240 VAC”. This embodiment uses un-boosted mains power, or any source of (120/240 VAC), to charge a group of storage elements in parallel connection which, after charging, are switched to a series connection to increase voltage to that required for defibrillation. As shown in FIG. 4, the raw mains electrical current 160 is the ordinary household current of 120/240 VAC. This current is used to charge a plurality of capacitors directly through the rectifier diode D1 180 (Diode D1 can be either a half wave or a full wave rectifier for faster charging) and the “charge or discharge” capacitor connection switches 181, 183, 185, 189, 197, 186, 197, all being shown in the “charge” position. These “charge or discharge” switches are represented as mechanical switches or relays for clarity, but it is to be understood that modern semiconductor switches such as SCRs and IGBTS and the like are often more advantageously used for this switching function and such use is well known in the art. FIG. 4 shows three storage capacitors, 182, 185, 197 for illustration purposes, but the actual minimum number of said capacitor storage elements will be determined by the required high voltage amplitude needed for successful defibrillation and the peak voltage of the raw alternating current input 160. Typically in the USA, this peak AC voltage is 120/0.707=170 peak volts. Thus, if it is desired to have a high voltage peak amplitude to deliver to the patient of 1700 volts, it is necessary to have at least 10 such capacitor storage elements due to the summing of their individual voltages when arranged in series. These capacitor storage elements are all initially charged in parallel, either simultaneously or in a specific order under the control of block 133 by using the switches positioned so as to charge each capacitor. Said switches are shown such that all capacitors are being simultaneously charged, but sequential or other order is achievable by selective positioning of said switches to include and exclude specific capacitor energy storage element's exposure to the charging voltage from 160 as rectified by diode 180. This ability to limit the number of storage elements being charged at any one moment is important in some circumstances, since it can be used to limit the required current to be delivered from the AC power source 160 and hence not overload the AC power source represented by 160. A major advantage of this embodiment of block 140 is that no transformer or voltage booster is required to charge the storage capacitors, thus reducing weight and complexity and allowing very rapid charging.

Once the storage capacitors are charged to the desired voltage, 170 volts in this example, the “charge or discharge” capacitor connection switches 181, 183, 185, 189, 197, 186, 197 are all switched to their opposite position and thus the high voltage energy is achieved by series connection of all storage capacitor elements upon reversal of the position of said switches. This bank of stored high voltage energy is connected through the high voltage wires 190 and 191 to the shock delivery and wave form shaping block 137 as previously described. It is to be understood that although in the illustration only three storage elements are shown being charged in parallel by raw current from household source of 120 VAC represented by 160 and through diode 180 in order to achieve the hypothetically desired 1700 volts, in actuality ten such capacitor storage elements, and their analogous switches must be charged to 170 volts in order to achieve this desired high voltage level. Using this design, any desired voltage can be achieved from the raw alternating current input simply by increasing or decreasing the number of storage elements which are charged from the input. Reducing the charging time can also be used to reduce the amplitude of the voltage stored in each capacitor and hence lower the sum amplitude when the capacitors are switched to be connected in series. Similarly, second or third banks of capacitors could be charged simultaneously or sequentially with that capacitor bank shown in order to supply one of the phases of a biphasic or triphasic shock or to permit very rapid sequential shocks to enhance defibrillation success. The circuit shows only those elements which are important to describing the overall function of the high voltage creation and energy storage functions of this embodiment.

This flexibility and economy of the high voltage creation and storage design shown in FIG. 4 is accomplished without the requirement for a transformer or other voltage multiplying device, and is enabled by of the use 120/240 VAC household current used to power the AED in this no internal-integral power battery embodiment. This novel approach to AED powering and high voltage generation allows smaller, lighter, and less expensive construction than prior internal-integral battery powered AEDs which all use relatively low voltage DC batteries as their sole source of power and therefore must include additional switching circuitry and step-up transformers to boost the chopped battery voltage to the high voltage required for defibrillation. Since the design and use and of such DC battery powered high voltage energy creation circuitry is well known in the art, and is in fact practiced by all battery powered photoflash units as well as all existing AEDs, such method of high voltage energy generation will be utilized in other embodiments of my AED, but will not further described due to its ubiquity.

In FIGS. 4, 5, and 6, the switches 198 and 199 represent a simple implementation of the power isolation block 138 shown in FIG. 3 and are shown as an electromechanical relay. It is to be understood that this optional patient isolation safety mechanism can be implemented with several different means, including semiconductor switches or relays and can be redundantly implemented or singly implemented as is shown. This understanding also applies to the same patient isolation function 138 and switches 198 and 199 shown in all other pertinent figures.

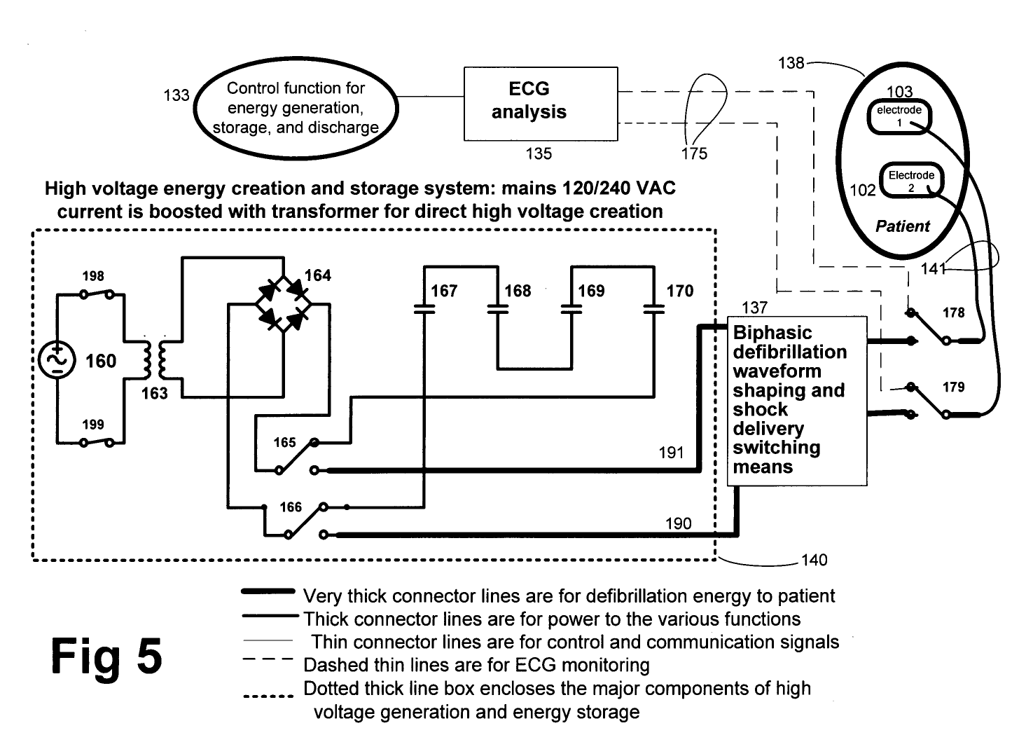

Looking next at FIG. 5, the means for high voltage creation and energy storage 140 is described and labeled as “mains 120/240 VAC is boosted using a step-up transformer for direct high voltage creation”. In this embodiment, as shown in the figure the raw mains electrical current 160 is the ordinary 120/240 VAC of household current. This current is first passed through a voltage step-up transformer 163 to directly achieve the maximum desired peak high voltage. In the case of 120 VAC input 160, if it is desired to achieve a voltage of 1800 volts peak, we must use a transformer 163 with a primary to secondary turns ration of 1800/(120/0.707)=10.6 that is the ratio of secondary to primary turns used in the step-up transformer must be in the order of 10.6 turns on the secondary high voltage side of the transformer 163 to every one turn of the primary side of the transformer which is connected to the mains current of 120 VAC in the USA. For countries which use 240 VAC as their mains current, the turns ration would be 10.6/2=5.3 since in that case the primary is at a voltage twice as high as in the USA. In many cases it will be advantageous to use higher turns ratios in transformer 163 and to terminate charging at the desired peak voltage, but prior to that peak otherwise possible with the higher turns ratio than required transformer. Basically, this design approach of higher turns ration than actually needed for desired peak voltage will reduce charging time substantially in this embodiment of 140 in FIG. 5.

This high voltage alternating current thus achieved by the step-up transformer 163 from the raw mains input is then full wave rectified by 164 for faster charging than a half-wave rectifier would permit (although a half-wave rectifier could be used to limit mains current consumption during capacitor charging), and said rectified current is used to charge a single capacitor or a plurality of serially connected capacitors connected by the “charge or discharge” switches 165 and 166, said switches being shown in the “charge” position in the figure and are represented as mechanical switches for simplicity, but it is to be understood that modern semiconductor switches are often more advantageously suited for this switching function. The figure shows four series connected storage capacitors, 167, 168, 169, and 170, but the actual minimum number of said capacitor storage elements will be determined by the required level of high voltage needed for successful defibrillation and generated by transformer 163 and the capacity and working voltage of the individual capacitors. In practice, the number of capacitors could be as few as 1 which provides the required storage capacity and working voltage for defibrillation or as many as 20, or even more, which in sum would deliver the required capacity and working voltage in combination. The illustration of 4 such capacitors is only one such possible implementation of block 140 in FIG. 5.

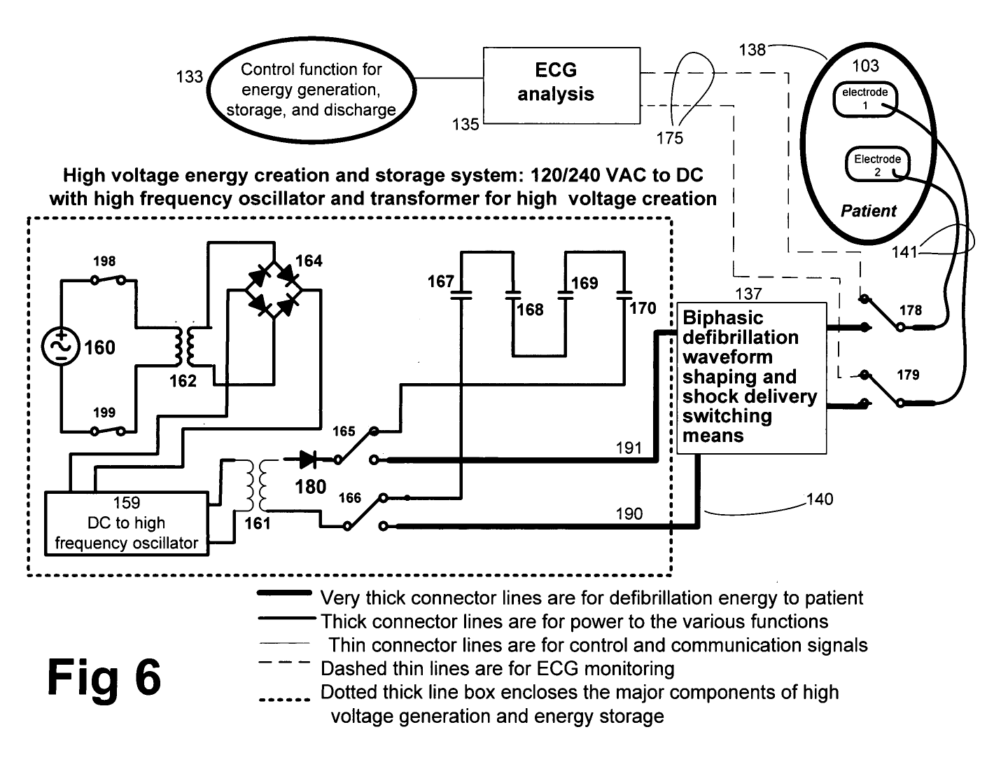

As can be seen in this FIG. 5, as well as in FIGS. 4 and 6, the high voltage created by the various means illustrated in the three embodiments illustrated is connected to the “wave form shaping and shock delivery block” 137 for ultimate discharge into the patient through electrodes 102 and 103 or alternative electrodes such as esophageal or cardiac electrodes or some combination of the three types of electrodes as shown in FIGS. 9a-9c. As previously described, the biphasic (or other multi-phasic waveforms) defibrillation waveform that is used in the inventive AED embodiments is created by the wave shaping and discharge block 137. As previously described, this functional block optionally implements an “H-bridge” energy source switch in those embodiments which use a single high voltage energy storage bank. This method delivers a portion of the remaining energy in the primary energy storage means after the first phase of the defibrillation waveform is delivered to the patient. This second portion of the waveform is sourced from the remaining energy in the high voltage energy storage means and is applied to the patient in reverse polarity as compared to the first portion of the waveform. This reversal of polarity is created by the well known “H-Bridge” long used for reversing motors, and which is a part of the functional bloc 137. The circuit shows only those elements which are important to describing the overall function of the high voltage creation and energy storage functions of this embodiment.

In an alternative method of delivering the reversed polarity second portion of the defibrillation waveform to the patient, a second separate energy storage means, typically a bank of one or more storage capacitors, arranged similarly to the arrangement of the primary energy storage bank illustrated in the FIGS. 4, 5, and 6 is utilized and is similarly charged as is the primary storage bank is charged, but optionally to a lower peak voltage. When applied to the patient through the appropriate switches contained in the block 137, the discharge of energy from the secondary energy storage means is of reverse polarity as compared to the energy discharged from the primary energy storage means. The choice of which of these two methods is used to create the opposite polarity energy delivery to the patient from and AED using a biphasic defibrillation waveform is determined by many design considerations related to size and cost and it is not important to specifically describe them further. Regarding the efficacy of the waveforms delivered, the two methods of creating the biphasic defibrillation waveform, with its reverse polarity second phase just described, are equally effective at defibrillation, since there is, as a practical matter, no difference in the phases and the energy actually reaching the patient between these two different methods of creating the two phases of the shock waveform.

Looking next at FIG. 6, the means for high voltage creation and energy storage is described and labeled as “120/240 VAC to DC with high frequency oscillator and transformer for high voltage creation”. This embodiment uses mains power, or any source of (120/240 VAC), and a transformer and bridge to create a DC current. This DC current is then chopped to create a high frequency AC current that is boosted by the transformer, then rectified and stored in one or more storage elements in serial connection (if more than one) to the desired voltage. As shown in the figure, the raw mains electrical current 160 is the ordinary 120/240 VAC of household current where said AC current is first passed through a transformer 162 to achieve the desired low voltage alternating current which is used to supply power the DC power supply and oscillator 159. The DC current output from said DC power supply in block 159 is subsequently converted within block 159 into a high frequency oscillation current which is delivered to the high frequency step-up transformer 161 where it is boosted to the high voltage required to charge the 4 storage capacitors 167-170. As in the other high voltage energy generation and storage embodiments of 140, the energy stored in said series capacitors is ultimately shaped into the desired biphasic (or multi-phasic) defibrillation waveform by 137 when it is delivered to the patient through electrodes 102 and 103 when the control system 133 determines that a shock is needed. It is to be noted, that this embodiment utilizes the DC power derived from the 120/240 VAC mains power in the same way that existing AEDs use the DC power from internal-integral batteries. The advantages of my use of 120/240 VAC in place of internal-integral batteries are many and include lower cost, faster recharge times, and greater reliability. The circuit shows only those elements which are important to describing the overall function of the high voltage creation and energy storage functions of this embodiment.

The various methods of generating high voltage from the input 120/240 VAC and storing it in capacitors as illustrated in FIGS. 4, 5, and 6 will each have advantages in terms of cost, size, weight, and charging speed which will make one of them most appropriate when combined with other design considerations for my AED in its various embodiments. For example, the embodiment of FIG. 6 is well suited to an AED that is optionally powered by external AC and external DC power sources as well as internal-integral batteries. However, the three approaches of FIGS. 4, 5, and 6 are functionally equivalent and are illustrated in some detail to demonstrate the great utility and flexibility that eliminating internal-integral batteries from my AED in these embodiments and powering the AED with ordinary 120/240 VAC household current provides.

New AED Adds Options for Internal and External Power Sources for Enhanced AED Reliability

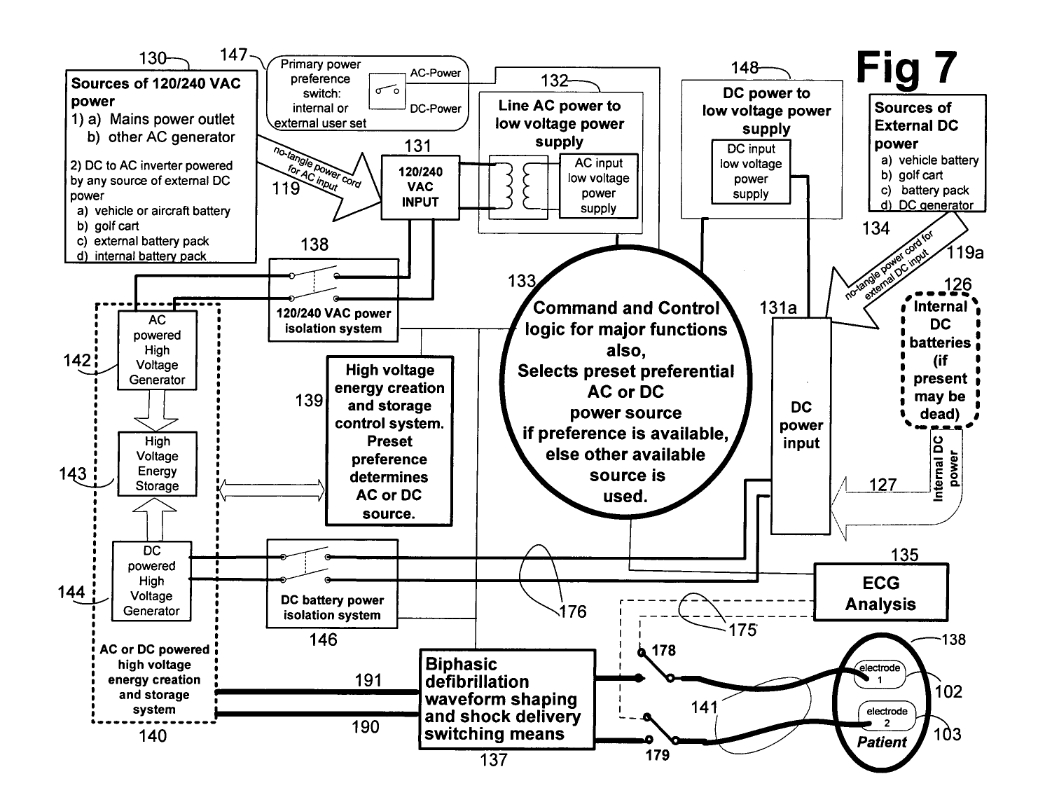

In FIG. 7 is a block diagram of the inventive AED similar to that shown in FIG. 3. In FIG. 3 the AED show was powered exclusively by 120/240 VAC, whether from mains power, a motor driven AC generator, or from a DC to AC inverter powered of off a source of external DC power all indicated in block 130. In FIG. 7, the AED design of FIG. 3 is enhanced to additionally permit operation of the AED directly from various external DC sources as shown in block 134 or optionally directly from internal-integral DC batteries schematically illustrated as block 126. The connection of the external DC sources 134 is through a no-tangle DC power cord 119a similar to the AC no-tangle power cord 119 except that 119a has a DC connector plug instead of the AC connector plug on 119. In those cases where a shorter power cord is applicable, a shorter cord is substituted for the no-tangle DC power cord 119a at a savings in cost and space. However, in the embodiment illustrated in FIG. 7, there are thus two such power cords 119 and 119a for accessing external AC and DC power, respectively, both available for available for use at any time, and one or both may optionally be plugged into their respective power sources simultaneously to provide AED power redundancy if desired. By using these external sources of AC or DC power, the optional internal-integral DC batteries, if present, are thus reserved for future use when external AC or DC sources may not be available or convenient. Additionally, this direct external DC powering enhancement present in this embodiment permits the operation of the AED directly from internal-integral batteries as well as from external DC power sources without the need for a DC to AC inverter as shown in block 130. Likewise, if the optionally present internal-integral batteries 136 are of the rechargeable type, they would be recharged by the external source of AC or DC power whenever the AED is connected to either type of external power source.

In FIG. 7 the high voltage generation and storage function 140 is expanded as compared to that in FIG. 3 so that high voltage generation is additionally provided by the DC powered high voltage generator 144 when operating on external or optional internal-integral DC batteries. Regardless of the source of DC power, such DC power is supplied from said DC source to 144 via connections 176 and through the DC isolation system 146. This low voltage DC battery to high voltage generation function is accomplished by 144 using the traditional method of converting the DC current to high frequency low voltage AC current and boosting this low voltage AC using a high frequency transformer to achieve the high voltage necessary, after rectification, to charge the capacitors in the storage block 143. This traditional method of conversion of low voltage DC current to rectified high voltage AC was previously illustrated more fully in FIG. 6 as blocks 159, 161, and 180 and is well known in the art of AEDs, electronic ignition systems, and photographic flash units as the preferred means used to create high voltage, for storage in a high voltage storage capacitor, from a low voltage DC input such as from an internal-integral battery or an external source of DC.

Since the AED design illustrated in FIG. 7 can use external DC sources 134 as well as internal-integral batteries 126 or external AC input as power for the AED, there is provided an isolation system 138 for the AC input and a similar isolation means for the internal or external DC power 146. Using these isolation systems, the control system 133 can at any time select the power input to 140 and can disconnect either or both sources in the event of a detected malfunction.

Also shown in FIG. 7 is the primary power preference switch 147 set at the factory or optionally by the user to select which power source is primary when both external AC and DC are both present simultaneously. Regardless of how 147 is set, the AED of FIG. 7 will function properly if any one of the three potential sources (external AC, external DC, or internal-integral DC batteries) are present, thus providing the greatest opportunity for successfully powering the AED and saving an SCA victim.

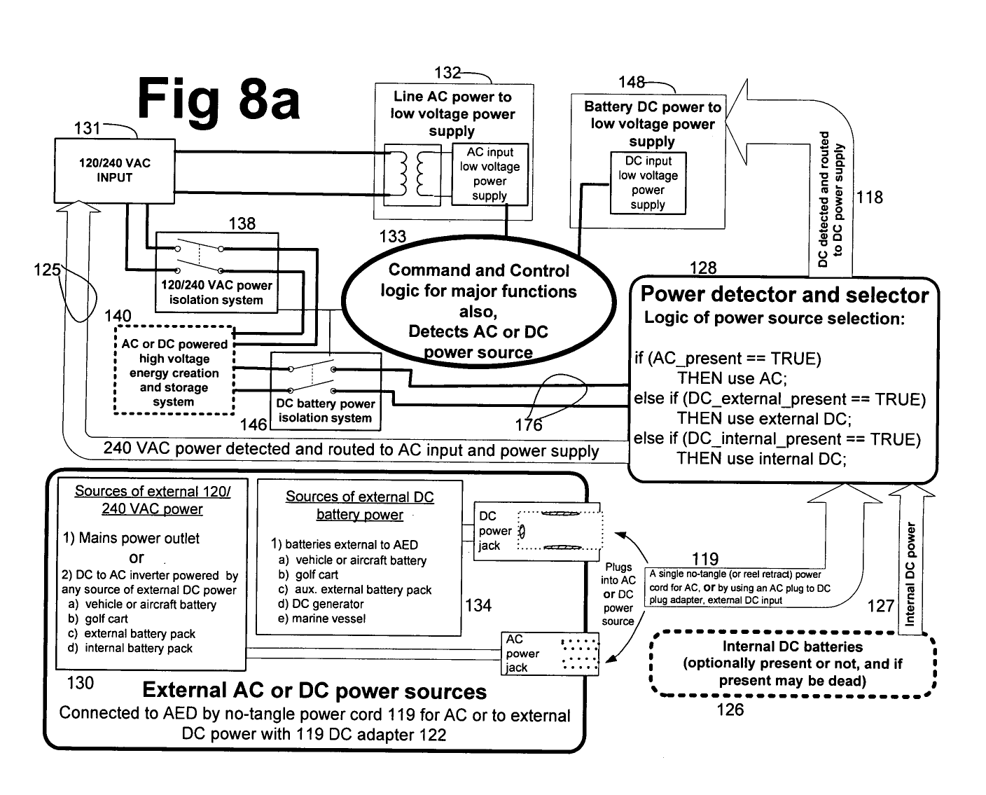

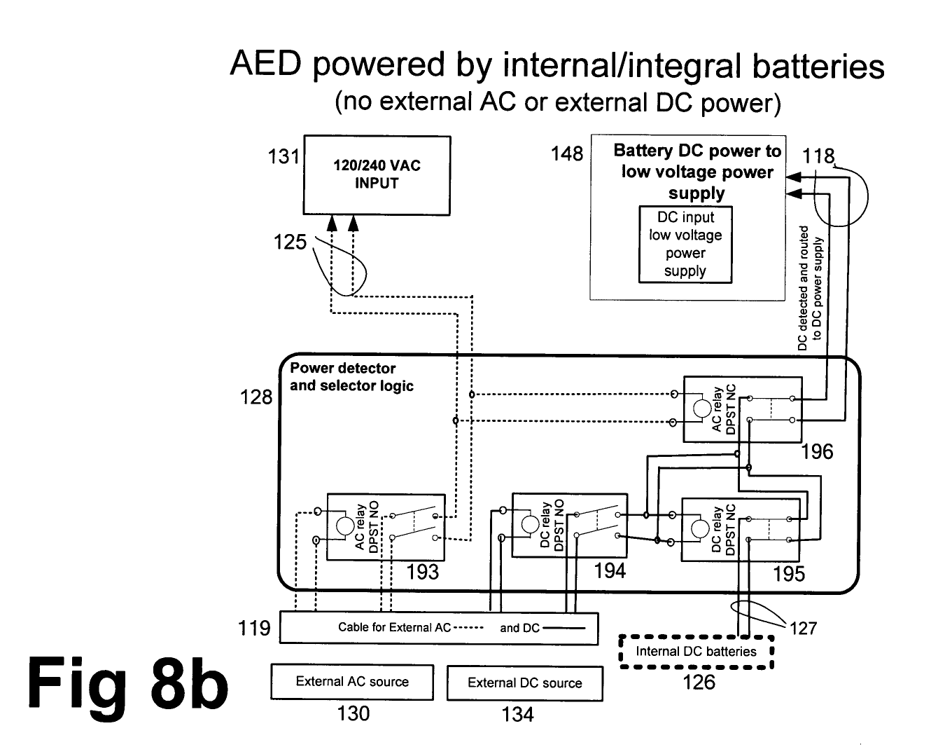

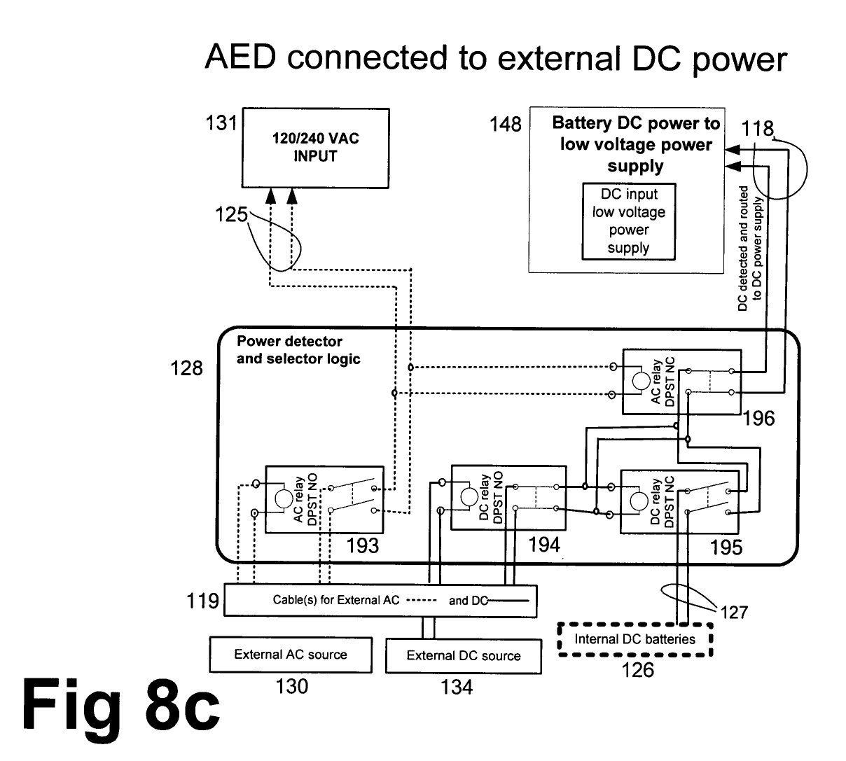

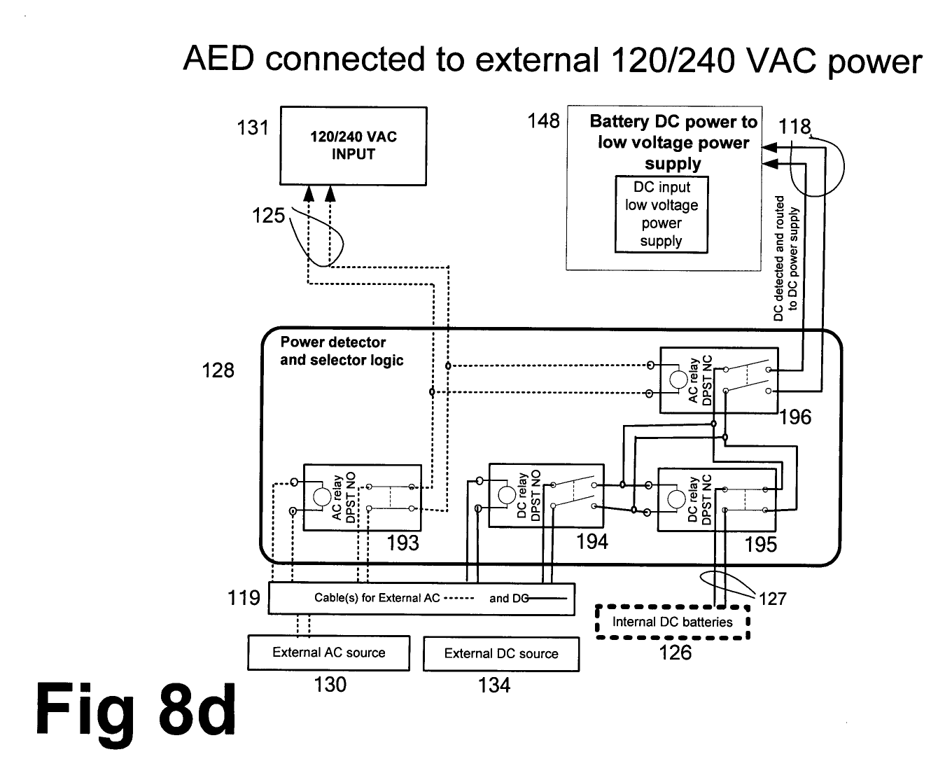

In FIG. 8a is shown a block diagram of an embodiment of the inventive AED similar to that shown in FIG. 7, except in FIG. 8a the functional blocks shown are restricted primarily to those functional blocks relating to the alternative powering sources of the multi-powered embodiment, namely internal-integral batteries 126, external AC power 130, and external DC 134 power and the power detection and selection logic 128 which determines which power source is actually used for powering the AED when more than one such source is present. The AED embodiment in FIG. 8a utilizes only a single no-tangle external power cord 119 to access either external AC 130 or external DC power 134. When the cord plug 116 is primarily designed for AC attachment as illustrated in FIG. 2 116, it is required that the AC to DC adapter 122 be attached to the AC plug (FIG. 2 116) before inserting it into an external DC socket such as the 12 volt power outlet or cigarette lighter power socket found in most motor vehicles. Thus the embodiment of FIG. 8a requires only one external power cord (optionally such external power cord is a single use, no-tangle assembly 119, a regular multi-use external power cord, or a multi use external power cord mounted on a self retracting reel) to access either an AC or DC source of external power. The AC plug to DC plug adapter 122 is required if the cord assembly 119 is fitted by default with an AC plug, and in the opposite case, a DC to AC plug adapter is required for AC power access if 119 is fitted by default with a DC plug.

Depending on what power is actually available to the AED powering embodiment of FIGS. 8a-8d, the power detector and selector block 128 automatically determines which source of power is primary and operates the AED on that source. One example of such power detect and select logic is illustrated in 128 in FIG. 8a and FIG. 8d and as shown uses AC 130 as the first choice if present and internal-integral batteries as the last choice. Such detect and select logic function 128 is active continuously and can switch from one power source to another power source automatically if the primary source becomes unavailable and another alternative source is available. Further, by utilizing a temporary energy storage element, such as an internal battery or an internal storage capacitor, the switching from one power source which has failed to a different, good power source, can be accomplished quickly and such that the AED is never without power, and such that the switch in primary power source by 128 does not appreciable delay the ECG analysis and defibrillation functions of the AED. This might be important for example when the source of external power, for example external AC 130, was initially present, but due to any one of many possible malfunctions, the external source of power becomes unavailable, in which case if another external source of power, for example external DC 134, is also connected to the AED would take over and power the AED. Or, in the case where there were no other external source of power connected to the AED, the optional internal-integral batteries 126 would immediately begin to power the AED at the failure of all of the external power sources 130 and 134.

The power detection and selection logic 128 can be implemented principally with electromechanical elements such as relays as illustrated in FIGS. 8b-8d, or principally with semiconductor elements or any combination of such elements. The method of connecting external power to 128 shown in FIG. 8a using only a single power cord 119 reduces the size of the AED enclosure (if said power cords are stored within the AED enclosure) as compared to that external powering method of FIG. 7 which requires and illustrates two power cords to access external AC and/or DC power sources. However, in another embodiment of the power detection and selection logic shown in FIGS. 8a-8d, both AC and DC power cords are present and only one connected to the appropriate external AC or DC power source, or both can be connected simultaneously used if it were desired to have the AED connected simultaneously to two external sources of power 130 and 134 for redundancy. Having a power detection and selection logic module 128 utilizing either one or two simultaneous external sources of power simplifies the power selection logic as compared to that in FIG. 7 where there is optionally also a user settable power priority selection switch 147. Such automatic power detection and selection circuitry 128 simplifies both AED electronic design and rescuer interaction during an SCA since power selection is automatic and there is no user interaction required to select the preferred power source.

Such detection and selection logic illustrated in FIG. 8a 128 can be implemented easily using relays as shown in FIGS. 8b-8d, but other semiconductor implementations are equally effective and may be optionally substituted. The switching elements 193, 194, 195, and 196 are shown in the figure as double pole single throw relays, wherein 193 and 196 are only actuated by AC and 194 and 195 are only actuated by DC. In FIG. 8b it can be seen that if no external source of power is connected through power cord 119, then, if present, the internal-integral batteries 126 are automatically selected as the AED's power source through the normally closed relays 195 and 196.

However, if either external DC 134 as in FIG. 8c, or external AC 130 as in FIG. 8d, (or both AC and DC simultaneously) are connected through 119 to the power detect and select logic 128, then the switching elements 193, 194, 195, and 196 switch to select external AC if connected or external DC 134 if external AC 130 is not connected to 128. When such external power connections are made by 128, regardless if whether AC or DC power is selected, the internal-integral battery 126 is opened by AC relay 196 or by DC relay 195 to prevent 126 from supplying power to the AED, although other circuitry (not shown) will still allow the internal-integral batteries 126 to be charged if they are of the rechargeable type.

More specifically, in FIG. 8d, external AC power 130 is detected by the AC activated relay 193 in the power detection and selection logic module 128 closes sending AC power via connections 125 to the 120/240 input module 131. Relay 193 also applies AC to activate AC relay 196, the AC relay 196 is thus opened to prevent applying power from the internal-integral DC batteries to the AED. Thus, the AC power 130 is routed to the AED's 120/240 VAC input module 131 and thus becomes the AED's powering source.

However, as illustrated in FIG. 8c, if external power cord 119 is connected to an external source of DC power 134, then DC relay 194 closes, applying DC activation current to the DC activated relay 195 which is thus opened to prevent applying power to the AED from the internal-integral DC batteries 126, and the external DC power source 134 becomes the AED's powering source via the normally closed connection of AC activated relay 196. If the external DC power 134 is of a voltage out of the range that is nominally supplied to the AED by the internal-integral DC batteries 126, then a DC-DC voltage converter must be used to convert the voltage of the external DC power 134 to a level close to the nominal voltage of 126 and thus suitable for powering the AED. Such a voltage converter is well known in the electronic art is not shown since it will not be needed in many cases and since its presence does not contribute fundamentally to the power detection and selection logic of block 128.

In all of the embodiments where external DC power is usable as a power source by a specific AED embodiment, it is to be understood that many acceptable means of connecting DC power to the AED are available and that the use, for illustration, of a cigarette lighter type of plug, while both simple and ubiquitous, is in no way the only acceptable means of such external DC power connection. In some circumstances, the external source of DC power might be coupled to the AED power cord with alligator clips or other such electrical attachment means. In other cases, the external power (AC or DC) might be wired directly to the AED via a suitable connector which is a part of the AED itself or even directly to the internal circuitry of the AED if the installation of the AED and its powering arrangements are to be permanent. This latter permanent or semi-permanent mounting and powering of an AED might be the case in an EMS vehicle or in a hospital specialty area where defibrillation is often necessary, and an AED or defibrillator is always present, such as an cardiac operating room or an electrophysiology laboratory where defibrillation is done many time a day.

It is an object of this invention to provide external power for my AED in all of its embodiments, regardless of whether traditional internal-integral batteries are present. Such external powering capability using external AC or DC power, available from the obvious and previously enumerated sources, greatly enhances the reliability and serviceability of an AED. However, for use in extreme cases an additional external power source contemplated by my AED is electrical power created by a human powered electrical generator. Such typically hand powered generators are well known in other arts and are used to power emergency telephones, lights, radios, and the like, but have not been used to power AEDs, since no existing AEDs accept an external source of power as does my inventive AED.

Though not specifically shown in the illustrations as an external source of AC or DC power, it is understood that my invention includes the use of external power generated by a human being supplying mechanical effort to create electrical power sufficient to power my AED for at least one defibrillation shock. There are many existing and commercially available hand powered electrical generators that will continuously output several watts of electrical power which if powering my AED as an external source of power would require activation for a minute or more, at that rate of energy production, to supply enough electrical energy for one shock, such shocks being typically at least 150 Joules or equivalently 150 Watt-Seconds. Nonetheless, in circumstances where no other internal or external power was available, this somewhat limited electrical power created by human effort would still be sufficient for defibrillating an SCA victim and could therefore be life saving in even the most extremely isolated circumstances. Hence the creation of an AED that is powerable by such human created power is an important feature of my externally powered AED invention. As a part of my invention, it is intended that such human powered generators could be included within the AED enclosure itself, thus becoming an internal, non-battery, AC or DC power source for those applications where it was important to have the human powered AC or DC generator included within the AED enclosure for compactness or environmental protection or other such purposes. In other circumstances, the human powered generator will be connected to the AED via a power cord, several of which have already been described and are suitable as are others.

New AED Adds Additional Electrodes Capability for Enhanced Defibrillation Effectiveness

As has been previously suggested, and referring again to FIGS. 9a, 9b, and 9c, my AED is optionally designed to accept and utilize not only the two surface electrodes traditionally used by all existing AEDs as well as manual defibrillators, but also to allow the addition, or substitution, of one or more additional electrodes such as additional surface electrodes, esophageal electrodes, or cardiac electrodes. It is well established in the scientific literature that electrodes closer to the heart are more likely to be successful in achieving defibrillation with the lowest possible energy and hence my inventive AED is designed to allow the use of alternative electrodes, alone or in combination with the traditional surface pad type electrodes 102 and 103 shown in various of the figures.

Looking again briefly at FIG. 3, the figure shows schematically the pad electrodes 102 and 103 on the ventral surface of the patient 138, such electrode placement is standard. In some cases however it is advantageous to place one of the electrodes directly over the heart on the ventral surface and one correspondingly behind the heart on the dorsal surface of the patient for enhanced defibrillation efficacy. This anterior and posterior body surface electrode configuration may well produce effective defibrillation even when the traditional placement has failed to defibrillate the patient. The disadvantage of this positioning is that it requires more time to place the electrodes and some amount of physical strength on the part of the rescuer to temporarily roll the patient such that the dorsal surface of the patient is accessible for posterior electrode placement. Although potentially a more effective electrode configuration for defibrillating an SCA victim, it is rarely used in first responder AED application.

However, there are additional, alternative electrode configuration methods enabled by my AED design that cannot be implemented with the existing AEDs. These optional additional alternative electrode types and electrode configuration sets can also be used to enhance defibrillation efficacy in difficult cases.

FIGS. 9a-9c illustrates three examples of such alternative electrode set configurations enabled by the design and construction of my AED. In FIG. 9a is shown an esophageal electrode being used as a part of the defibrillation electrode set. It is connected to connector 68 of the AED electrode connection panel 58 in place of one of the two body surface electrodes 103 which in this electrode configuration set is moved over to connector 64. This electrode configuration could be used to provide defibrillation in instances when using only the two surface electrodes in the standard anterior positions alone are not sufficient to deliver enough focused electrical energy through the heart for successful defibrillation. Notice that in this case shown in FIG. 9a the two surface electrodes 102 and 103 are moved closer together and placed almost directly over the heart in order to focus the current directly through the heart as the current passes between the body surface electrodes 102 and 103 and the electrode 54 in the esophagus directly behind the heart. The advantage of this specific electrode configuration is that the esophagus is posterior to and very close to the heart and the surface electrodes are placed directly over the heart, thus, the shock vector is directly through the heart and is very short in distance, both of which prevent energy wasting caused by the defibrillation energy spreading out in the volume conductor represented by the patients thorax. Such energy wasting by volume conductor spread occurs when the shock vector is long and not focused directly through the heart, as is the case to some extent in the traditional AED electrode placement on the body surface of the patient. In the traditional placement, both electrodes are on the ventral chest surface, with one surface electrode in the right sub-clavicular position and the other surface electrode on the left side, at roughly the level of the anterior axillary line, and just below the estimated lower margin of the heart. Thus the more advantageous geometry of the configuration in FIG. 9a achieved by using an esophageal electrode 54 and repositioning the two surface electrodes closer to the heart (only one surface electrode could be optionally used), might well be successful in cardiac defibrillation even when the traditional electrode set configuration of two surface electrodes on the ventral chest surface fails to defibrillate successfully.

The alternative electrode configuration sets shown in FIG. 9a and 9b are easily set up by a trained rescuer and may be lifesaving when rescuers are faced with a failure to defibrillate using conventional surface electrode placement. Similarly, these alternative electrode configurations might be used by the EMS personnel who arrive to assist the first responder rescuer after being summoned by a call to “911” and find the patient refractory to AED defibrillation using the conventional electrode placement. This esophageal to surface electrode configuration has experimentally produced success when other electrode configurations have failed, even after many minutes of fibrillation.

The unique design of my AED to include a rescuer accessible and configurable electrode connection block 58 with extra sets of electrode connectors 60, 62, and 64 for connecting electrodes to the initially positive side of 58, and extra sets of electrode connectors 68, 70, and 72 for connecting electrodes to the initially negative side of 58, enables this additional electrode connection and configuration flexibility which will increase the efficacy of defibrillation in certain circumstances, including severe cardiac injury and patient obesity.

It is to be understood that the terms initially positive and initially negative with respect to 58, and the associated multiple electrode connectors, refers to the polarity of the first phase of a biphasic or other multi-phasic defibrillation waveform or to the single phase of a monophasic waveform. Further, though shown with a total of 6 connectors for creating multiple electrode configuration sets, any number of such electrode connectors could be used if so desired to increase or decrease the number of potential electrode connections. Additionally, in the AED embodiments which enable rapid sequence, multiple defibrillation shocks using different electrodes for each of a sequence of shocks, the AED electrode connection panel 58 would be replicated for each additional shock in the sequence beyond 1. Thus, if an embodiment enabled three rapid sequence shocks, each shock potentially using a different electrode set, there would be three such AED electrode connection panels as illustrated in all of the relevant figures as 58. Thus in this latter embodiment example, there would be 58a, 58b, and 58c electrode connection panels present where 58a would be used to configure the electrode set for the first of three sequential shocks, 58b for the second of three sequential shocks, and 58c for the third shock. Where one or more electrodes are used for more than one of the sequential shocks, a short jumper, or other such connection means, can be used to connect a given electrode to more than one of the AED electrode connection panels. Notice also that where rapid sequence sequential shocks utilize the same set of electrode set for each shock, there is no requirement for the additional AED electrode connection panels beyond a single one 58 as illustrated in FIGS. 9 and 1.

In FIG. 9b, the defibrillation electrode configuration is such that only body surface electrodes 103b, 103a, and 102 are used. However, in this case, instead of using only two surface electrodes as is done with all existing AEDs, three surface electrodes are used, with the additional surface 102 electrode being placed directly over the heart and used to better focus the energy through the heart than is possible with only two surface electrodes. Note that 103b and 103a are connected to 68 and 70 in the initially negative group of connectors in the electrode connection panel 58. Better results using this electrode configuration set, (as well as all of the others), are possibly obtained by reversing the sense of connection so that, in this example, electrode 102 would be connected to the initially negative block of connectors and conversely, 103b and 103a would be connected to 60 and 64 for example in the initially positive group of connectors in 58. This defibrillation enhancement using a reversal of initial polarity of the electrode set is a possibility with any set of electrodes, and such reversal should be tried if failure to defibrillate is consistently present in the rescue attempt of an SCA victim. This ability to reverse the sense of the defibrillation electrode set, in order to potentially enhance a failed defibrillation, is unique to my AED since all others do not permit this potentially advantageous reversal due to their design and construction which limits both the number of electrodes to two and fixes their connection polarity sense to the one which cannot be changed.

It is to be understood that the electrode arrangements in FIGS. 9a-9c are for illustration purposes and do not constitute the only potentially useful electrode arrangements possible. These, and other electrode configurations, are easily implemented with my AED, which in the illustrated embodiment provides complete flexibility via connection block 58 to arrange the shocking electrodes as desired by the rescuer. However, the AED will typically be delivered to the owner with only the two surface electrodes 102 and 103 connected as illustrated in FIG. 1, but in the field, an advanced rescuer could reconfigure completely the electrode configuration set for enhanced results in difficult cases by changing the electrode sense and by adding or substituting electrodes to provide the best results, as for example, in patients who are obese or who have otherwise been refractory to previous attempts at defibrillation using the traditional two surface pad electrode configuration. This flexibility to configure alternative electrode sets and sense is not provided by any existing AEDs and represents a major enhancement to the utility and effectiveness of my design.

Looking now at FIG. 9c, and as an example of another potential electrode configuration achievable with the inventive AED, a cardiac electrode is shown being used with a single surface electrode for defibrillation of a patient. Typically, such a configuration would be used in a patient that has a preexisting existing cardiac electrode and has developed fibrillation requiring defibrillation (either atrial or ventricular defibrillation). If the cardiac electrode is attached to the atrium instead of the ventricle, then in this configuration the AED would be being used in an automatic atrial defibrillation mode. With this direct atrial connection the AED could be used to alert the user and permit manual or automatic cardioversion of the atrial fibrillation if so desired. This mode of direct atrial connection and AED use would be particularly useful in patients recovering from cardiac surgery, since about one-third of such patients develop atrial fibrillation post operatively that must be detected and treated by atrial defibrillation. This atrial fibrillation detection and defibrillation process could be accomplished using my AED since it is designed to permit the connection of various electrodes, including atrial and ventricular cardiac electrodes. Similarly, if the cardiac electrode is a ventricular electrode, then ventricular fibrillation would be detected by the AED and defibrillation would be ventricular defibrillation which is the traditional fibrillatory arrhythmia associated with SCA but which is detected by surface electrodes only in all prior AEDs.

Continuing with FIG. 9c, the cardiac electrode is shown as the initially negative electrode, and the single surface electrode placed over the heart is the initially positive electrode, but this configuration could be reversed if desired and other cardiac or surface electrodes added to the electrode configuration set if desired.

With the flexibility of my AED invention, all of these electrode set configurations, as well as the many other possible combinations not being show in the figures, are also easily achieved. In all three illustrations shown in FIG. 9, it can be seen that the defibrillation shock is delivered between the alternative electrode and one or more surface electrodes. The inadequacy of using only two surface electrodes is particularly a problem in SCA subjects who are very obese or who have been in VF for a prolonged time, and the esophageal electrode, alone or in combination with surface electrodes or cardiac electrodes may successfully defibrillate these patients, even when defibrillation attempts using only two surface electrodes have failed completely. Similar advantages also apply to the cardiac electrode(s) and the three body surface electrode configurations.

As the scientific literature on defibrillation proves, successful defibrillation of the heart is never assured and often requires modifications of traditional techniques. Only my AED permits such modifications of techniques by enabling different electrode configuration sets, rapid multiple shocks through the same or different electrode sets, and a delayed shock option to delay the delivery of the defibrillation shock until the frequency and/or the amplitude of the detected ventricular fibrillation ECG waveform indicates a statistically advantageous time to deliver the shock.

The group of electrode connectors in the electrode connection block 58 in FIG. 1 and FIG. 9 labeled in the figure as the “AED Electrode connection panel” is, in the illustrated embodiment, a part of the AED front panel itself and positioned by design so as to be readily accessible to the user in the process of rescue. However, it may be advantageous that these multiple patient electrode connectors be made remote from the body of the AED itself by using a group of conductors contained within one or more wire cables to connect said patient electrode connectors 60, 62, 64 and 68, 70, 72 to the electronics of the AED itself. Such extension of and optional positioning of the actual electrode connectors remote from the AED unit itself may, in certain circumstances, make connection of any of the patient electrodes more easily and more quickly achieved by the rescuer.

Most patients, who are not obese, will be defibrillated by one or more 150-360 Joule biphasic waveforms, as are variously used by current AEDs as well as my inventive AED, if applied within the first two minutes after a SCA. However, the ability of my AED to accommodate alternative electrodes and multiple electrode configuration sets and to allow polarity reversal of the electrodes is both novel and very valuable in difficult cases, particularly where trained, professional EMS personnel are in attendance or when used in the hospital by nurses and physicians. Such difficult defibrillation cases may be due to prolonged duration of fibrillation before attempted defibrillation, underlying cardiac pathology, severe obesity, or other complicating aspects of the attempted rescue. Such multiple and alternative types of electrodes as here disclosed as being usable with my AED will result in additional lives saved as compared to the existing AEDs and their use of only two surface leads. The four extra electrode connectors shown in 58 (that is, four extra connectors in addition to the two needed for the two traditional surface electrodes) shown in FIGS. 9a-9c are present, but unused when only a total of two electrodes are being used. These extra connectors may be covered with a removable cover or protective plug to prevent any possible confusion on the part of an untrained rescuer or any possible contact with the unused connectors during shock delviery. However, said unused electrode connector covers or plugs, when removed, will provide direct access to the additional connectors for attachment of additional or substitute electrodes to create alternative and generally more advantageous electrode sets for defibrillation than the traditional two surface electrodes used by all of the current AEDs

Three of many such different alternative electrode sets are illustrated in FIGS. 9a-9c. It is to be understood that the illustration of three such alternative electrode sets for defibrillation of patients using my AED are in no way illustrative all of the potentially advantageous electrode sets constructed using some combination of surface, esophageal, and cardiac electrodes and my invention is inclusive of these additional combination electrode sets, such sets comprised of any combination of body surface, esophageal, and cardiac electrodes.

New AED Provides Integrity and “Freshness” Assurance as well as Environmental Protection

Referring again to FIG. 1, the AED enclosure 101 is shown with one surface opened, said opened surface being the lid or cover of the AED 89. As shown, this cover 89 is hinged at one edge 90 such that lid 89 is openable, but not fully removable from the rest of the AED enclosure 101. In another embodiment, the hinge at lid edge 90 would be absent so that the lid or cover 89 could be totally removed. In either of these embodiments of the lid or cover of the AED, it is possible to bridge the seams between each edge of the lid and the main portion of the AED with a frangible label such that the frangible label would be disrupted if the lid was, for any reason, opened or totally removed, depending on the embodiment of the AED enclosure lid or cover. Thus the frangible label, if disrupted, would be an indication of prior use or prior opening and hence an indication that the AED might not be serviceable and should be discarded or refurbished before being put back into service. This freshness indicator, that is, the frangible label bridging the lid 89 and the body of the AED enclosure 101, could also be constructed such that it contained on its visible surface a written date or date code whereby said date indication could be indicative directly of the expiration date of the AED or alternatively of the date of manufacture or refurbishment, from which the date of expiration could be inferred, or both, as well as an indication that the AED was not guaranteed fresh if the frangible label was breached. In this mode of use, the frangible label is used totally to assure freshness and indicate date of required disposal or refurbishment.

In either mode of attachment of the AED lid 89 to the body of the AED, that is either being hinged or being fully removable, it is also advantageous to provide a sealing means such that the lid is sealed to the body of the AED enclosure 101 by some means, such sealing to prevent entry of possible environmental contaminants such as water, moisture, dirt, dust, or other such possible contaminants that could potentially degrade the performance of the AED. One such means of sealing the lid 89 to the AED enclosure body 101 is to provide a gasket which covers the mating edges of the lid with the body 101 of the AED. Thus when the lid 89 is closed and pressed firmly against the body 101 of the AED and affixed in that closed position by suitable means such as a closure latch 91 and 88 or alternatively by an external band or tape securement capable of retaining the lid 89 in apposition to the body 101 thus compressing the gasket to seal lid 89 to the body 101, thus providing a tight seal which will exclude environmental contamination.

Another method of sealing the lid 89 to the body 101 of the AED to exclude environmental contamination and which does not require the gasket just described, is to circumferentially place a sealing tape around the entire AED unit when the lid 90 is closed on the body 101 of the AED enclosure, whereby said sealing tape bridges and covers the small gaps between each edge of the lid 89 and the body 101 of the AED enclosure, and thus prevents the entry of environmental contaminants. The frangible seal to indicate freshness described above could be so constructed and positioned that it could easily accomplish both the function of indicating freshness (or tampering) as well as sealing the AED to prevent the entry of contaminants. Other such methods of sealing the lid to the body of the AED enclosure are also possible and are considered equivalent in function to the described methods.

Either of these contamination prevention sealing methods are applicable to both the hinged and the fully removable styles of AED enclosure covers. Additionally, when the AED enclosure is thus sealed, it may also have the above described frangible label affixed to it such that when the environmental seal is broken, such action will also breach the frangible AED freshness label as a further indication that the sealed AED has been opened and hence its freshness, and its readiness for proper function, can no longer be guaranteed.

If either of these methods of protective sealing are completely air tight, then the sealed AED unit will optionally use a breather port or valve which will allow small amounts of air to enter or exit the sealed AED. These small amounts of air passage for pressure equalization may be necessary if the change in atmospheric pressure from the site of manufacture or refurbishment to the site of use is greater than a certain amount, estimated to be 2000 feet and 5000 feet of elevation difference. Thus it is anticipated that in those cases where the sealing mechanism is completely air tight such a pressure equalization device will be required in many cases. Such pressure equalization devices which allow small amounts of air to pass but prevent moisture from passing are well known and include devices such as the breather included in many sealed food packages such as coffee and others or the manual pressure equalizer screw found in ridged, airtight, gasketted plastic or metal storage and transport boxes.

However in the simplest case, it is anticipated that during AED sealing a small air vent will be intentionally created to prevent the need for a specific pressure equalization device. For instance, a small breather hole in the gasket or the tape seal described above would be sufficient for such equalization and prevent the need for a specific device. Alternatively, the AED enclosure could be made sufficiently compliant that pressure equalization would be achieved to the extent required simply by bulging or compression of the sealed AED enclosure; or the AED enclosure could be made sufficiently strong that the difference in internal and external pressures would not result in any failure of the seal or the AED enclosure.

An alternative method of sealing my AED for tampering indication and for contamination prevention is an over-wrap cover of protective material FIG. 10e 27 and is fully described below.

New AED Has Many Visibility Options for Selectable Environmental Integration

Now referring to FIGS. 10a-10e, the AED enclosure 101 is so shaped that it can be easily positioned and displayed vertically or horizontally on a table, shelf, book case, or wall mount bracket so that it is ready for immediate use, much as a fire extinguisher is often wall or shelf mounted. Further, said AED is optionally fitted with a sealed protective cover 27 to create a waterproof and contaminant exclusion barrier as illustrated in FIGS. 10c and 10e wherein AED 101 is covered with a protective barrier. The protective cover is additionally optionally fitted with a tear tab FIG. 10e 26 or other equivalent means for assisting the user in rapidly opening the sealed protective cover 27 when the AED is needed for application to a SCA victim.

Whether in its un-covered configuration or covered with the protective over wrap 27 and opening tab 26 configuration, the new AED is equipped on one or more of its surfaces with external decoration or indicia affixment means, such as hook or loop fasteners (Velcro), slots or pockets, or other such affixment means to provide, optionally, decoration using one or more of several different external placards, indicia, or pictures to allow user selection and affixment of said external display placard means. These external decorations are such that they create the desired degree of AED obviousness or subtlety for blending into a room in the home or workplace in which the AED is stored and positioned ready for use. Several different examples of such positioning and environmental blending are shown in FIGS. 10a-10d.

In FIG. 10a the AED 101 is very obvious and would be obvious to anyone seeing it. This would be appropriate indicia and positioning for an AED that is to be positioned where persons not familiar with the positioning of the AED would see it and use it if needed. Somewhat less obvious is the AED positioned and placarded as shown in FIG. 10b. In FIG. 10c the AED 101 is, within its outer protective cover 27, positioned and placarded as a painting or photograph and is substantially less obvious than in the two previous cases. Similarly, in FIG. 10d the AED 101 is positioned and placarded as a book with only a subtle indication that it is not actually a book, but an AED ready to save a life.

It is to be understood that many variations on these illustrated mounting, storage, and obviousness options are available and the illustrations of FIG. 10 show only a few of the many options that are possible to encourage positioning the AED for maximum access in the home or workplace. These options are all uniquely enabled by my AED 101 design and its AED over-wrap cover 27 when present.

Alternative Power Supply Modules for External Powering of an AED that is Normally Powered by an Internal-Integral Battery.

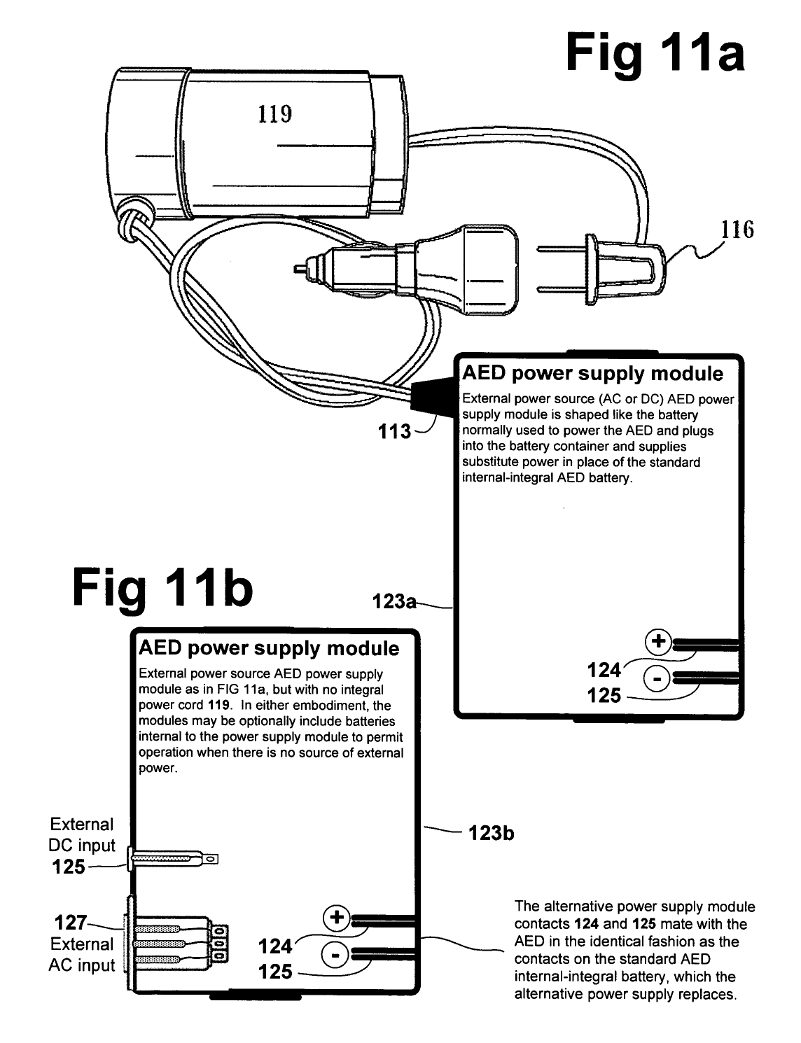

In FIG. 11a is one embodiment of a “battery substitute power module” and comprises AC and DC power conversion circuitry within the battery shaped housing 123a, an AC/DC power cord assembly 119, AC plug 116, and DC adapter 122 for cigarette lighter socket insertion thus permitting an existing AED to be powered from external AC or DC sources. This external power source AED power supply module substitutes for the standard internal-integral AED battery. It is shaped like the AED specific internal-integral battery so that this power module can fit properly within the AED battery slot and make the required power connection to the AED, thus supplying proper DC power to the AED in place of the standard internal-integral DC AED battery. This substitute power supply thus converts a normally internal-integral battery powered AED to an externally powered AED. In this embodiment, the power cord enters the power module with a strain relief 113 and is shown as a non-removable cable. Removable cable is also possible. In certain cases, a conventional shorter power cord is preferable to the long no-tangle cord assembly 119 and can be substituted.

FIG. 11b shows an external power source AED power supply module shaped like the AED's normal internal-integral battery as in FIG. 11a, but with no integral power cord 119. This module permits external power to be used with an AED which is originally designed to use battery power only, just as in FIG. 11a. In this embodiment, the power module is fitted with AC 127 and DC 125 connectors in place of the single attached power cord assembly 119 shown in FIG. 11a. The power modules of both FIGS. 11a and 11b also can optionally contain internal batteries that can be used to power the AED when no external source of power is available.

In the embodiment shown in FIG. 11b, the external AC and DC power source's connecting cords connect to the power supply module using power source specific connectors, one for AC input 125 and one for DC input 127. In either embodiment, the modules may be optionally include batteries internal to the power supply module to permit operation when there is no source of external power.

Application of Features of Inventive AED to Existing Internal-Integral Battery Powered AEDs

Many of the reliability and versatility advantages of my AED over existing AEDs are achieved by various AED design embodiments which permit the use of alternative power sources whether or not there is an internal-integral battery as compared to the single source of power provided for in existing AEDs, which is solely internal-integral batteries. It is an object of this invention to expand the advantages of optional external power to existing AEDs and FIGS. 11a and 11b illustrate another aspect of my invention which comprises a substitute power source for existing AEDs which are designed to use user replaceable internal-integral batteries. The substitute AED power source shown in FIGS. 11a and 11b as 123aand 123b respectively is designed to be the same size and shape as the original AED battery and to duplicate the original battery's electrical connections to the AED 124 and 125 such that when 123 inserted into the AED in place of the native AED battery, this substitute AED power module makes the appropriate electrical connections to successfully power the AED using external AC or DC power sources, just as if it were a fully charged battery. The power module 123a is fitted with an external power cord assembly 119 which contains a long power cord used to connect the AED power module 123a to the source of external power, whether such external power is AC or DC power. In the case of using external DC power as from a vehicle or the like, the DC adapter 122 would be used to convert the two pronged AC power plug connector 116 to a connector shape and style suitable for connection to the selected source of external DC power, here shown as 122 which is designed to plug into a cigarette lighter power jack socket.

The other end of the power cord non-detachably joins to the AED power module through the strain relief 113 and supplies external AC or DC to the circuitry of the module which converts the power input to the proper DC output which supplies the power for the AED as if it were a battery that never runs down with use. The power thus provided to the AED has its origin external to the AED thus enabling AED operation for reliable external sources of power in place of the less reliable internal-integral batteries. In one embodiment of this substitute power module, the AED substitute power module will also contain internal-integral batteries which will power the AED should no external power sources be available. In this way, the substitute power module when used with existing AEDs, which are normally exclusively powered by internal-integral batteries, permits those AEDs to be powered by external power sources and optionally internal-integral batteries contained within the substitute power module itself. This increased flexibility in powering existing AEDs extends their reliability and utility and permits repeated use without depleting the internal-integral batteries if such are present in the power module.

In the case of inclusion of internal-integral batteries within the alternative power module, and depending on the size and capacity of the included batteries, it may be necessary to extend one or more dimensions or the overall shape of the alternative power module shown in FIGS. 11a and 11b as 123a and 123b respectively to provide sufficient space for such inclusion of the internal-integral batteries in addition to the power supply circuitry required to create the proper DC voltage needed by the AED into which the module is inserted or attached. However, the size and shape of that portion of the substitute power module 123a and 123b which fits into the slot in the AED normally occupied by the battery must remain sufficiently identical to that portion of the battery it is replacing to permit secure physical retention and good electrical contacts from the alternative power module contacts 124 and 125 to that portion of the recipient AED that engages the power contacts 124 and 125. Other than this constraint on the shape of the alternative AED power module there is no specific size or shape constraint and it may be made larger overall than the internal-integral battery it replaces if need be or desired.

In one extremely simple embodiment of an alternative power module, the alternative power could simply be an additional battery which is connected in parallel with the original battery or is switch selectable as the primary in case the original battery fails or becomes weak for any reason. This embodiment could optionally not have the external AC or DC power capability illustrated in FIG. 11, thus creating an alternative power module where the alternative power is additional batteries, thus giving redundancy to the item most likely to fail in existing AEDs. Obviously, many combinations are possible and all result in greater reliability of function of existing AEDs.

Referring now to FIG. 11b, in certain applications it is not necessary, or even desirable, to have the long, no-tangle power cord assembly 119 as shown in FIG. 11a, and in such cases, a simple AC or DC power cord is utilized rather than the longer cord 119. Specifically, the alternative power supply module 123b in FIG. 11b does not have the no-tangle external power cord input 119 as shown supplying external AC or DC power to the alternative power module 123a in FIG. 11a. Instead, in the embodiment shown in FIG. 11b, the power supply module 123b which, as 123a in FIG. 11a, fits into the normal AED battery compartment to permit operation from external power, has two external power input connectors 125 and 127. The input 125 is an input connector jack for attaching a cable from an external source of DC power, such as a DC power generator or the battery of a motor vehicle, while 127 is an input connector jack for external 120/240 VAC input. The DC and AC connectors illustrated are those traditionally used for connecting external sources of DC and AC power to various electronic devices, but other sizes, shapes, and types of connectors are equally serviceable in this application. As is the case in the embodiment shown in FIG. 11a, in the embodiment shown in FIG. 11b there can be internal batteries contained within the power supply module for powering the AED when no external source of power is available, with or without an expansion of external size depending on the size of the batteries included.

In this way, the existing AED can be converted to have the powering options of my inventive AED. That is, it may be optionally powered in any of three ways using the power supply module of FIGS. 11a or 11b: 1) internal batteries within the power supply module itself, 2) external DC power, or 3) external 120/240 VAC power. This ability to be powered from any of three possible sources will virtually eliminate the situation where the AED is unserviceable due to lack of adequate power.

New AED Enables a New AED Service Business Model and Method

The invention of my new AED enables and encourages the need for an inventive new method of doing business in the promotion, sales, and refurbishment of AEDs, largely converting the traditional product business into an AED access business. This new business method is almost essential for the inventive AED but is also applicable as well to existing multi patient use AEDs, particularly when such traditional AEDs are fitted with the alternative power module just described.

Since many embodiments of my new AED are designed to be single use, and used principally with external 120/240 VAC or external DC power, (instead of internal DC power supplied by replaceable internal-integral batteries), it requires a long power cord for connecting to ordinary mains current or external DC power, previously full described. This power cord, when not utilized with a retractable reel, is packed into a power cord container previously described and illustrated in FIG. 2, much like a parachute is packed into its container for a single jump use. Such single use no-tangle cord packing as illustrated in FIGS. 2a-2b, is specifically designed for single use and repacking of the cord is required to be accomplished at the factory after such single use or after the expiration of the specified shelf life to assure it will function without tangling when deployed. Similarly, the patient electrodes are single use items which must be replaced after use or expiration of their shelf life.

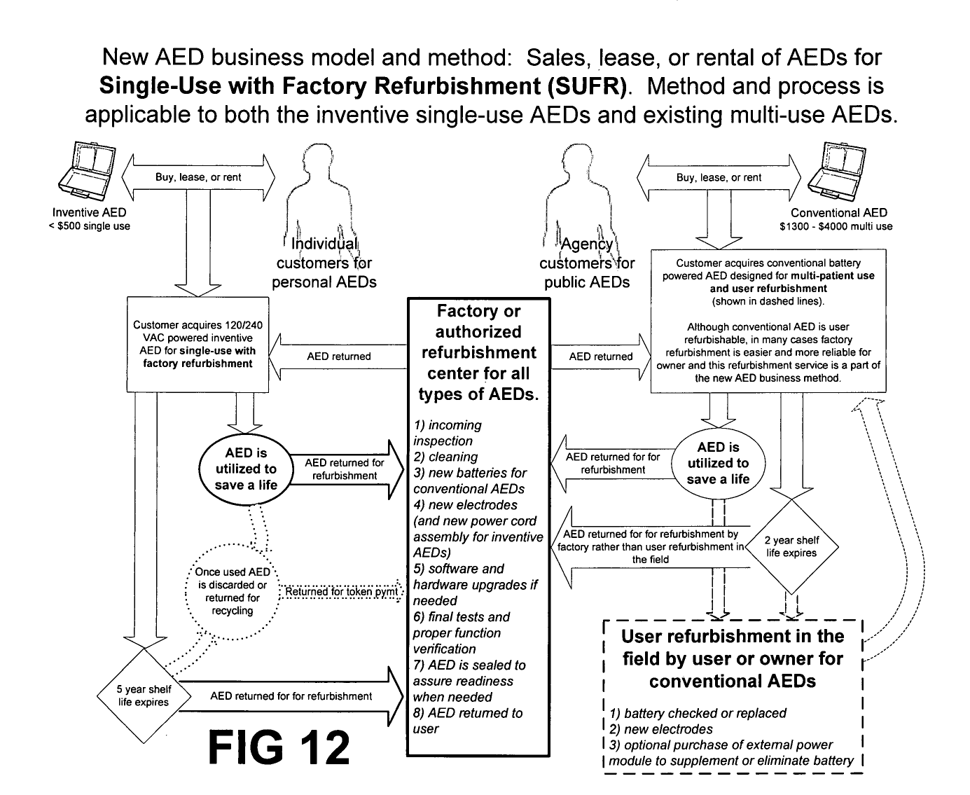

My new AED business model or method is schematically illustrated in FIG. 12 and is referred to as “Single-Use with Factory Refurbishment” (SUFR). This new business method largely converts the distribution of AEDs for home and workplace use to an “AED access service”.

People who desire to have AED access can subscribe by buying or renting AED a low cost AED for placement in their homes or workplaces. This initial payment thus provides them AED access for a specified period of time, most typically the shelf life of the AED, which in the case of the inventive AED is designed to be 4 years or greater due to the elimination of internal-integral batteries as the primary source of power. Thus the cost of AED access is a one time payment for the specified term or alternatively to a very low monthly payment.

Central to this AED access business model is the refurbishment center represented by the block labeled “Factory or authorized refurbishment center . . . ” in the center of FIG. 12. This refurbishment block receives inventive AEDs from customers on the left, said inventive AEDs originally designed for single use, or it receives conventional AEDs from customers on the right, such conventional AEDs originally designed for multi patient use and user refurbishment in the field or which are which are potentially fitted with the alternative power module for existing AEDs.

Referring to the left side of FIG. 12, it is seen that the new AED business method, here described for the first time, is one in which the inventive AED, and hence personal access to it, is acquired through purchase or rental by a customer for a period of access, typically the shelf life, or for a single-use which ever comes first. Thus the inventive AED typically enters a “personal AED” mission where the anticipated possible single-use is targeted at “Private Access” as compared to “Public Access” typical of existing AEDs.

The inventive AED, after being utilized for life saving, or after expiration of its shelf life, is returned to the factory or authorized refurbishment center shown in the center of the figure, since (unless it is specifically a user refurbishable embodiment of this invention), it must be factory refurbished before subsequent use. In most embodiments of the inventive AED, said AED is designed to function only one time and is designed to prevent multiple uses after a specified short period of time after first use without first being returned to the factory for refurbishment. The exception to this limitation on repeated use without requiring factory refurbishment is obviously the embodiments where user refurbishment is deemed desirable by the specific AED application environment or ownership and is so permitted by the AED design and configuration.

In all single use embodiments, the expiration date or life is indicated on the unit at time of manufacture or refurbishment as described above. Additionally, in one embodiment, there is an optional battery powered timer and shelf life expiration annunciation function FIG. 3 129 to alert the user it is time for refurbishment of their AED in case they have forgotten to periodically check the date of expiration. At the refurbishment center, whether the AED has been used or has simply expired its shelf life, the AED is completely refurbished utilizing the steps shown in the figure, such refurbishment steps comprising cleaning and inspection, replacement of worn or expired parts such as any internal-integral batteries if present, the power cord assembly, and the patient electrodes. Likewise, the refurbishment process requires the performance of any required software or hardware upgrades to the AED, plus testing and validation of proper function, and finally resealing of the AED to prevent or indicate prior use or tampering as well as environmental protection. The thus refurbished AED is then returned to the original owner.

However, in many cases it may be desirable to return to the owner a like AED, which is newly refurbished, but which is not actually the exact same AED unit which was sent to the refurbishment center by the owner. The immediate shipment of a like, but not the identical, AED to the owner provides the owner a less than 24 hour turnaround time with a refurbished replacement AED if they desire, thus reducing their time without AED access to no more than a day. Since the AEDs thus exchanged are functionally and operationally identical, the owner is provided an equivalent AED unit which again has at least a 4 year shelf life. Since like the new unit, the refurbished replacement unit is sold for onetime use only and is sealed at the factory or refurbishment center and guaranteed as if new, whether the owner/user is returned their very same AED is generally of no consequence or concern since they are essentially buying not specifically and AED but access to and AED for a specified period of time. For shelf life expiration refurbishment needs, an equivalent refurbished AED can be pre-shipped to the owner before they send in their expired AED. This pre-return replacement of the customers AED provides two major advantages to the customer: 1) the AED owner is never without access to a personal AED, and 2) the AED needing refurbishment can be returned in the same shipping carton used to deliver the refurbished replacement AED. Both of these advantages enhance the levels of security, reliability, and convenience that my new AED access service business method is designed to provide.

For a variety of reasons, an AED owner may not want to pay the refurbishment fee for a refurbished AED which has been used or is now expired its shelf life. Said owner may simply discard the used or expired AED without further consideration. However, as a part of the AED access service business model, the manufacturer will pay the previous subscriber a small sum for any returned AED in refurbishable condition. By doing this fully refurbishable AEDs are retained in the overall AED unit pool, and can be then be refurbished and sent to other subscribers who need a refurbished unit or sent to initial subscribers who wish to purchase initially a used but fully refurbished unit at a slightly reduced price as compared to a totally new unit. Since the AED unit is being provided under this business method for defined period of time access and for single use only, and since it is guaranteed for 4 years or more of shelf life, there is no quality or functionality penalty suffered by a person who chooses to subscribe to the AED access service by buying, leasing, or renting a “used but refurbished to new condition AED” at the reduced price for such used, by fully refurbished, AED units. Some subscribers will prefer subscribing by buying a truly new single use AED but many will prefer to subscribing to the AED access service by acquiring a fully guaranteed refurbished unit and thus saving some money.

My business model a fundamental previously overlooked by all other AED designers and marketers: it fundamentally comes down to the fact that lay persons do not want to own an AED per se, what they want is access to an AED with a at a low cost and with a long maintenance free shelf life . . . just in case they may need it sometime . . . just like homeowners, automobile or life insurance. My new AED access service business based on a single use with factory refurbishment (SUFR) AED provides for this desired AED access at the lowest possible cost to the subscriber and is a major breakthrough in fostering immediate AED access for the greatest possible number of people in their homes and workplaces.

The new AED access service distribution business model described above is applicable not only to my inventive AED, but is also largely applicable to existing and future conventional AEDs. Conventional AEDs meaning those AEDs which use internal-integral, user replaceable batteries, and which are designed for multiple patient use and user refurbishment. Currently all existing AEDs are conventional AEDs. These conventional AEDs are often owned by groups or agencies which may have no specific training in the refurbishment, testing, and validation of proper function of AEDs after use or expiration of their shelf life. As the right half of FIG. 9 schematically illustrates, my new AED business model also anticipates the needs for conventional AED refurbishment, whether such need for refurbishment is due to actual use or to expiration of the much shorter shelf life of existing AEDs. As previously described, these shorter shelf lives of conventional AEDs is because they all contain internal-integral batteries as their only power source. Additionally, most of the conventional AEDs sold to date are for public access and professional use and hence are not as likely to be converted to a single use subscription service as is my inventive AED.

By utilizing the refurbishment services provided in my new business method for AED distribution and refurbishment, existing AED owners who prefer to not be responsible for the refurbishment, testing, and validation of proper AED function can return their used or shelf life expired AEDs to the AED refurbishment center for complete refurbishment, including the hardware and software upgrades recommended by the AED's original manufacturer. This refurbishment could be on a subscription basis for multiple refurbishments or on a one-time basis. In this way, my business model will allow owners of conventional AEDs to become a part of a method and service of AED management which I have termed “Single-use with factory refurbishment” (SUFR) and through which the business method offers subscription AED access. By utilizing the refurbishment of my business model, current owners of conventional AEDs eliminate the expense and liability of AED self refurbishment and processing when done by their own staff. In some cases, the availability of these services provided by my new business method will eliminate the need for one or more in-house refurbishment staff and hence result in considerable cost savings to agencies placing such AEDs for public access or other such purposes.

In summary, the new AED access service business model for AED distribution here described comprises selling, leasing, or renting AEDs at low cost (because of the novel low cost and high reliability, and long shelf life design of the inventive AED) for a single-use or at least 4 years, which ever comes first. If, subsequent to either event, it is desired by the owner that said AED be refurbished, the AED can be refurbished for less than one half of the original cost of a new AED. Used, but refurbished, AEDs are sold to subscribers for less than new ones, but greater than the refurbishment cost alone since there is no “exchange unit” being provided by the new subscriber. However, such used but refurbished AEDs carry the same guarantee as a new AED and can be refurbished after use or expiration for the same price as an AED acquired new by a new subscriber. The full development and commercial deployment of this new personal AED access service business will gradually transform the majority of AED placements from those placed through the current AED product business of selling multi-use AEDs to customers with in the field user refurbishment, to where the majority of AEDs being placed are placed through my AED access subscription service business, where such subscribers purchase an AED access subscription service by one of several methods and finance such AED access subscription by either onetime or periodic payments as is done with many other services to which individuals subscribe: cable TV, Internet access, telephone, etc. After shelf life expiration or a single use, the subscriber must return the AED for refurbishment and pay an additional fee for such refurbishment unless the subscription is of the specified uses or unlimited use type for which the subscriber pays more initially.

FIG. 13 is a side-by-side comparison summary of:

1) my new AED and its associated business method for providing private AED access and refurbishment of both inventive and conventional AEDs, as compared to,

2) all currently existing AEDs and the currently existing AED product sales business method.

FIG. 13 lists and summarizes many of the improvements previously described in the figures and text of this patent application and brings many of these improvements together for direct comparison with current AEDs and the old AED product business model. It is to be understood however that some categories of features of my new AED and business model are not listed in the chart since there is no equivalent category in existing AEDs.

Conclusions, Ramifications, and Scope

The above descriptions of my new AED and various of its embodiments, and the description and methodology of my new private access, single use AED business model-method for AED distribution and refurbishment, contains may specifics as to design, features, and utility of my AED and method of operation of my new AED business model. These specific descriptions, and the various figures use to further illuminate certain aspects of my invention, should not be construed as limiting the scope of the invention, but merely as providing descriptions, illustrations, and examples of some of the presently preferred embodiments of my invention. Therefore, the foregoing is considered as illustrative only of the principles of the many and various aspects of the invention. Further, since numerous modifications, combinations, and changes will readily occur to those skilled in the art, it is desired to not limit the invention to the exact AED construction, operation, and business model shown or described; accordingly, all suitable modifications and equivalents may be resorted to, falling within the scope of the invention.

What is claimed is:

1. An AED that performs all of its intended functions without the use of any battery supplied electrical power.

2. An AED which is powered exclusively by external electrical power.

3. An AED which is powered by external electrical power when connected to said external electrical power, but said AED being powered by internal-integral batteries if said external power is not connected to said AED at time of intended use.

4. An AED which is powered by alternating current.

5. An AED as in claim 4 which is powered exclusively by alternating current.

6. An AED as in claim 5 wherein said alternating current is 120/240 volt alternating current.

7. AED which is powered by alternating current when said alternating current is connected to said AED, but wherein said AED is powered by internal-integral batteries if said alternating current is not connected to said AED at time of use.

8. An AED as in claim 7 wherein the change in said ADE powering from said alternating current power to said internal-integral battery power occurs automatically if the source of alternating power is lost for any reason.

9. AED which is powered by internal-integral batteries if said internal-integral batteries are present and if said internal-integral batteries contain sufficient power for proper operation of said AED and otherwise said AED is powered by an external source of power.

10. An AED as in claim 9 in which said external power is alternating current.

11. An AED as in claim 9 in which said external power is direct current.

12. An AED as in claim 9 in which said external power is at least one of a source of direct current and a source of alternating current.

13. An AED which is powered exclusively by external direct current.

14. An AED which is preferentially powered by external direct current, but wherein said AED is powered by internal-integral batteries if said external direct current is not connected to said AED at time of use.

15. An AED which is preferentially powered by external power, but is powered by internal-integral batteries if said external power is not connected to said AED at time of use.

16. An AED which is powerable by an external source of power wherein said external source of power is connected to said AED using a power cord.

17. An AED as in claim 16 wherein said power cord of at least 3 feet in length.

18. An AED as in claim 16 wherein said power cord further comprises a means for packaging and storing said cord such that when deployed for use said cord will not tangle.

19. An AED optionally powered by alternating current wherein said alternating current electrical power is generated by the physical efforts of a human being without the assistance of other motive power.

20. An AED as in claim 19 wherein said human powered AC electrical power generator is enclosed within the AED except for the actuation means grasped by the human being supplying the sole motive power.

21. An AED which is powerable and functional without the use of any internal-integral battery.

22. An AED as in claim 21 which is powered by external direct current powering means wherein said external direct current powering means is electrically connected to said AED by conductor means, said conductor means providing electrical continuity between said AED and said external direct current powering means.

23. An AED as in claim 22 wherein said independent external direct current powering means is a battery.

24. An AED as in claim 22 wherein said independent external direct current powering means is a battery within a vehicle.

25. An AED as in claim 22 wherein said independent external direct current powering means is a direct current electrical power generator.

26. An AED as in claim 25 wherein said direct current electrical power generator is itself powered by the physical efforts of a human being without the assistance of other motive power.

27. An AED as in claim 26 wherein said human powered direct current electrical power generator is itself enclosed within the AED enclosure means with the exception of the portion grasped by the human being supplying the sole motive power to the direct current generator.

28. An AED that contains an integral-internal battery for powering its operation and which additionally provides connection means and circuitry means for optionally powering said AED from an external power source instead of powering from its internal-integral battery.

29. An AED as in claim 28 where in said internal-integral AED battery is capable of being charged by power from said independent external power source when said AED is conductively connected to said independent external power source.

30. An AED as in claim 28 where in said independent external power comprises battery power from at least one of an external battery pack, a motor vehicle, a truck, a car, a golf cart, an aircraft, a water craft, and the like.

31. An AED as in claim 28 where in said independent external power comprises external 120/240 VAC power.

32. An AED as in claim 28 wherein said independent external power source comprises at both external 120/240 VAC power and external battery power from at least one of an external battery pack, a motor vehicle, a truck, a car, a golf cart, an aircraft, a water craft, and direct current generator.

33. An AED as in claim 22 where in said external power is created by a solar power panel.

34. An AED as in claim 22 where in said external power is power created by a human powered generator.

35. An AED with internal-integral AED batteries which additionally provides means for external power sources connections and circuitry means to permit full AED functioning when connected to and powered by one or more of said external power sources regardless of the state of the internal-integral AED batteries, said external power sources including at least one of batteries removably affixed to the AED externally, separate non-affixed portable batteries external to the AED, motor vehicular batteries, aircraft electrical power, electrical generator power, marine electrical power, and 120/240 VAC power of any origin.

36. An AED with no internal-integral AED batteries which provides means for external power sources connections and circuitry means to permit full AED functioning when powered by one or more of said external power sources, said external power sources comprising at least one of batteries removably affixed to the AED externally, separate non-affixed portable batteries external to the AED, motor vehicular batteries, aircraft electrical power, electrical generator power, marine electrical power, and 120/240 VAC power of any origin.

37. An AED as in claim 36 wherein said 120/240 VAC power is obtained from a DIRECT CURRENT to AC inverter means connected to an external battery means.

38. An AED as in claim 37 wherein said external battery means is located in a motor powered vehicle.

39. An AED as in claim 37 wherein at least one of said direct current to AC inverter means and said external battery means is removably affixed to the enclosure means of said AED.

40. An AED as in claim 39 wherein at least one of said direct current to AC inverter means and said external battery means is permanently attached to the enclosure means of said AED.

41. An AED which is powerable from at least one external power source means wherein AED connection to said external power source means is achieved with a power source connection means, said connection means being sufficiently long to reach from an external power source means to the patient.

42. An AED as in claim 41 wherein said AED power source connection means is greater than 1 foot in length.

43. An AED as in claim 41 wherein said AED power source connection means is less than 300 ft in length.

44. An AED as in claim 43 wherein said power source connection means is a power cord stored on a retractable reel means for rapid deployment and automatic retraction and rewinding after use and in preparation for next use, said automatically retracting reel means preventing tangling of the power cord while in storage and while being extended for use and retracted after said use.

45. An AED as claimed in claim 43, where said power source connection means is contained by a power source connection means container means which prevents power source connection means from tangling when said power source connection means is in storage and when said power source connection means is extracted from said container means and extended for the purpose of connecting said AED to an external power source.

46. An AED as in claim 45 wherein said power source connection means container means is built into and is integral with the AED enclosure means.

47. An AED as in claim 45 wherein said power source connection means container means is separable from the AED enclosure means and which remains electrically connected to said AED.

48. An AED as in claim 45 wherein said power source connection means is coiled in said power source connection means container means.

49. An AED as in claim 45 wherein said power source connection means is folded in said power source connection means container means.

50. An AED designed so as to permit only a single patient episode of use.

51. An AED as in claim 50 wherein said AED is factory refurbishable after said single patient episode of use so as to be usable on an additional patient only after each such refurbishment after use.

52. An AED as in claim 50 wherein said AED is powered exclusively by internal-integral batteries.

53. An AED as in claim 51 wherein said AED is powered exclusively by internal-integral batteries.

54. An AED as in claim 50 wherein said AED is powered exclusively by external electrical powering means.

55. An AED as in claim 51 wherein said AED is powered exclusively by external electrical powering means.

56. An AED as in claim 50 wherein said AED is powered exclusively by external 120/240 VAC powering means.

57. An AED as in claim 51 wherein said AED is powered exclusively by external 120/240 VAC powering means.

58. An AED as in claim 50 wherein said AED is powered by exclusively external direct current powering means.

59. An AED as in claim 51 wherein said AED is powered by exclusively external direct current powering means.

60. An AED as in claim 50 wherein said AED is powered optionally by at least one of an external direct current powering means and an external 120/240 VAC powering means.

61. An AED as in claim 51 wherein said AED is powered optionally by at least one of an external direct current powering means and an external 120/240 VAC powering means.

62. An AED as in claim 50 wherein said AED is powered by internal-integral batteries.

63. An AED as in claim 51 wherein said AED is powered by internal-integral batteries.

64. An AED as in claim 50 wherein said AED is powered by at least one of internal-integral batteries, external 120/240 VAC powering means, and external direct current powering means.

65. An AED as in claim 51 wherein said AED is powered by at least one of internal-integral batteries, external 120/240 VAC powering means, and external direct current powering means.

66. An AED as in claim 50 wherein multiple patient use is prevented by internal circuitry means which allows determination by said AED that said AED has been used previously and hence said AED is inhibited from additional use.

67. An AED as in claim 51 wherein multiple patient use without factory refurbishment is prevented by internal circuitry means which allows determination by said AED that said AED has been used previously and hence said AED is inhibited from additional use without prior refurbishment

68. An AED designed for single use as in 67 wherein multiple use is prevented only if said additional use is attempted after a specified allowable period of multiple use, said allowable multiple use period being timed from the time of first use.

69. An AED optionally powered by internal-integral or external power sources which contains an internal battery exclusively and totally dedicated to powering an internal clock means and an alerting means, said clock means keeping the time since last refurbishment of said AED, and triggering an alerting means to alert the user when said elapsed time is greater than the specified shelf life, said alerting being indication that there is a need for said AED to be refurbished due to shelf life expiration.

70. An AED as in claim 69 where in said shelf life timing and alerting battery is also used to power a testing means for the AED's internal-integral power battery and to trigger said alerting means if said AED internal-integral power battery has failed or is low on power.

71. An AED powered by electrical power source external to said AED, said electrical power source being created by an electrical generation means, said electrical generation means comprising a rotary electrical generator, said generator being rotated by a rotation means, wherein said rotation means comprises mechanical power supplied by a human being.

72. An AED as in claim 71 wherein said rotary electrical generator produces direct current which is used to power said AED.

73. An AED as in claim 71 wherein said rotary electrical generator produces AC current which is used to power said AED.

74. An AED powered by an external power source, said AED comprising circuitry means for internally disconnecting said external power source from the electronic circuitry of said AED if said AED circuitry determines that there is a fault which might result in malfunction of said AED.

75. An AED as in claim 74 wherein means for disconnecting said external power from said AED is at least one of a relay and a semiconductor switch.

76. An AED as in claim 74 wherein said fault is any voltage measured by said AED circuitry which does not fall within expected parameters at any time during AED operation.

77. An AED as in claim 74 wherein said fault is any voltage measured through electrodes attached to the patient by said AED circuitry which does not fall within expected parameters at any time during AED operation.

78. An AED powered by an external AC power source which includes means for electrically isolating the external AC power from the operator and the patient.

79. An AED as in claim 78 wherein said isolation means comprises at least one transformer.

80. An AED as in claim 78 wherein said isolation means comprises at least one transformer for isolating the AC current that is used for creating the high voltage required by the defibrillation shock and at least one transformer which is used to create the low voltage alternating current used to create the low voltage direct current which powers all other functions of the AED.

81. An AED as in claim 78 wherein said isolation means is a single transformer with multiple windings such that at least one transformer winding is used for isolating the AC current that is used for creating the high voltage required by the defibrillation shock and at least one transformer winding which is used to isolate and to create the low voltage used to create the low voltage direct current which powers all other functions of the AED.

82. An AED powered by external AC power which utilizes a step-up transformer means to increase the voltage of the AC power applied to the AED to a level higher than said AC voltage powering the AED, said higher voltage thus created being used by additional circuitry means to create the highest voltage required by the AED.

83. An AED powered by external AC power which utilizes a step-up transformer to directly increase the voltage of the AC power applied to the AED to the level of the highest voltage required by the AED without additional voltage boosting circuitry being required.

84. An AED powered by external AC power which contains circuitry means to convert said external AC power to direct current power internally to the AED which is then utilized to create the highest voltage required by said AED.

85. An AED powered by external AC power which contains a step-up transformer to boost the voltage of said external AC power to a higher voltage, said step-up transformer boosting the voltage of said external AC power to a level substantially less than the maximum voltage level required by said AED.

86. An AED powered by external AC power which contains circuitry means to convert said external AC power to high voltage without the use of a step-up transformer.

87. An AED as in claim 84 wherein the circuitry means for converting said external AC power to high voltage required for defibrillation comprises a charging means for charging a group of capacitors in parallel connection and after said charging of said capacitors, a capacitor switching means for switching said group of capacitors from said parallel connection to a serial connection such that the total voltage across said serial connection is sufficient for defibrillation purposes.

88. An AED powered by external AC power which contains circuitry means to convert said external AC power to high voltage without the use of a transformer in the high voltage creation process.

89. An AED powered by external AC power wherein said AED contains circuitry means to convert the voltage of said external AC power a lower voltage, said lower voltage being subsequently used to power both the high voltage creation circuitry and all of the other electronic functions of the AED without the use of a transformer in the high voltage creation process.

90. An AED as in claim 81 wherein said circuitry means is a step-down transformer.

91. An AED as in claim 89 wherein said lower voltage is converted to a DC voltage, said DC voltage subsequently being used to power both said low voltage circuitry and said high voltage creation circuitry.

92. An AED which optionally utilizes a plurality of electrodes, wherein said plurality of electrodes being greater than two.

93. An AED as in 92 wherein said plurality of electrodes are all body surface electrodes.

94. An AED which utilizes at least two electrodes wherein at least one of said electrodes is not attached to the body surface of the patient.

95. An AED as in claim 94 in which said non body surface electrodes are one or more esophageal electrodes.

96. An AED as in claim 94 in which said non body surface electrodes are one or more are cardiac electrodes.

97. An AED comprising a plurality of electrode connector means for connecting a plurality of electrode means to said AED wherein said plurality of electrode means is any combination of body surface electrodes, cardiac electrodes, and esophageal electrodes and wherein said plurality of electrodes comprises at least two electrodes.

98. An AED which optionally utilizes a plurality of electrode means, said plurality of electrode means comprising a user selected combination of any of number of surface, esophageal, and cardiac electrodes as may be attached on the body surface of the subject or within the body of the subject to be defibrillated by said AED, wherein each electrode means of said combination of electrode means is connected to said AED each using one connector means of a plurality of electrode connector means which are a part of the AED.

99. An AED as in claim 98 wherein said AED electrode connection means for a user selected combination of patient electrodes is a plurality of connector means integral with the AED.

100. An AED as in claim 98 wherein said AED electrode connection means is rendered remote from the AED enclosure itself by one or more conductors such that said AED electrode connection means for the selected combination of patient electrodes is at a distance remote from said AED itself, said connecting wires allowing electrical contact between the assorted electrodes attached to said connection means and the AEDs electronic monitoring and shock delivery circuitry contained within said AED.

101. An AED wherein after said AED establishes the presence of ventricular fibrillation, delays delivery of the defibrillation shock, said defibrillation shock delivery delay being intended to optimize defibrillation shock timing by analyzing the evolving pattern of at least one of the amplitude and the frequency of the ECG of the ventricular fibrillation and delaying shock until such analysis indicates that the heart is most likely to be defibrillated by the shock.

102. An AED as in claim 101 wherein said delay before shock delivery is enforced to be less than 30 seconds.

103. An AED as in claim 101 wherein said shock delivery is delayed until the frequency of the ECG fibrillation waveform measured in cycles per second has decreased to less than a specified number of cycles per second at which time the defibrillation shock is delivered.

104. An AED as in claim 101 wherein said shock delivery is delayed until the peak amplitude of the ECG fibrillation waveform measured in microvolts has increased to more than a specified level of microvolts at which time the defibrillation shock is delivered.

105. An AED as in claim 101 wherein said shock delivery is delayed until the earlier of a) the frequency of the ECG fibrillation waveform measured in cycles per second has decreased to less than a specified number of cycles per second and b) the duration of the interval of measurement of the ECG fibrillation waveform in cycles per second is greater than a specified number of seconds.

106. An AED as in claim 105 wherein said measurement interval is greater than 2 seconds and less than 60 seconds.

107. An AED as in claim 101 wherein said defibrillation delay is terminated as soon as the frequency of the ECG fibrillation waveform has decreased to a local minimum and is beginning to increase again after reaching said local minimum frequency of the ECG fibrillation waveform.

108. An AED as in claim 101 wherein said defibrillation delay is terminated as soon as the amplitude of the ECG fibrillation waveform has increased to a local maximum and is beginning to decrease again after reaching said local maximum amplitude.

109. An AED which is optionally able to deliver multiple shocks to the patient in rapid succession, said shocks being delivered such that a maximum of 5 seconds elapses between each such successive shock to the patient.

110. An AED as in claim 109 wherein the delay between successive shocks is between 1 nanosecond and 5 seconds.

111. An AED as in claim 109 wherein a fixed number of multiple shocks are rapidly delivered to said patient without user interaction and without said AED attempting detection of said patient's heart rhythm until after the last shock of said fixed number of multiple shocks have been delivered to said patient.

112. An AED as in claim 109 which is able to deliver multiple shocks to the patient in rapid succession, wherein said AED, after each shock, analyzes the patient's ECG to determine if another shock is needed to defibrillate said patient, and delivers said additional shock if needed to defibrillate said patient, wherein this cycle comprising shock delivery, post shock patient rhythm analysis, and if additional shock is needed then delivering said additional shock is repeated until defibrillation is accomplished.

113. An AED as in claim 1 12 wherein said repetitive patient analysis and shock delivery cycle repeats until the earlier of a specified maximum number cycles has been performed or defibrillation has been achieved, at which time the repetitive patient analysis and shock delivery cycle is terminated.

114. An AED as in claim 109 wherein said multiple shocks are delivered through the same electrode sets during each of the multiple shocks, hence repeating the same shock configuration and shock direction vector for each of shock of said multiple shock sequence.

115. An AED as in claim 109 wherein said multiple shocks are delivered through different electrode sets, such that each sequential shock in the multiple shock set is delivered through a different electrode set that is not identical to the electrode set used during the preceding shock, and hence the shock direction vector changes for each shock in the multiple shock sequence as compared to the previous shock in the sequence.

116. An AED which is powered by external power means and which does not utilize an ON/OFF switch and thus said AED is in its ON condition whenever external power is applied by connecting said AED to said external power source.

117. An AED as in claim 116 where said external power means is at least one of a source of external alternating current electrical power or an external source of direct current electrical power.

118. An AED as in claim 117 wherein said source of direct current electrical power is a source of 12 volts direct current.

119. An AED as in claim 117 wherein said source of alternating current electrical power is a source of 120/240 VAC.

120. An AED which utilizes an ON/OFF switch, said ON/OFF switch being placed in the ON position by the manufacturer or refurbisher before the AED is shipped to the customer, said switch ON/OFF being in the ON position ensuring that said AED will be active immediately after being connected to external power, and said ON/OFF switch additionally allowing said AED to be subsequently switched to OFF if desired and subsequently to ON again if so desired by the user.

121. An AED comprising additionally an automatic power detection and selection means whereby said AED is powered by at least one of 120/240 VAC, external direct current power, and internal-integral AED battery according to a pre established priority of power selection for powering said AED, said AED being successfully powered if at least one of said potential power sources is present and connected to said AED..

122. An AED comprising additionally an automatic power detection and selection means such that said AED is powered by 120/240 VAC power if said AED is connected to an external source of said 120/240 VAC power, else said AED is powered by external direct current power if said AED is connected to a source of said external direct current power, else said AED is powered by an internal-integral AED battery if said internal-integral AED battery is present and electrically connected to said AED.

123. An AED as in claim 122 wherein the priority ordering of the potential powering sources for said AED is reversed in order, such that said AED is powered preferentially by the said internal-integral AED battery if present and connected, else powered by external AC if present and connected, else powered by external direct current if present and connected.

124. An AED as in claim 123 wherein the priority ordering of the external powering sources is reversed, wherein if internal-integral AED battery power is not present, then the AED is powered by external direct current power if said power is connected to the AED, else said AED is powered by external alternating current if connected.

125. An AED as in claim 122 wherein the priority ordering of the external potential powering sources for said AED powering is reversed in order, such that external direct current power would be selected for AED powering before selecting AC power if both were present and the internal-integral power would be selected if no external power were connected to said AED.

126. An AED as in claim 125 wherein the priority ordering of the potential powering sources for said AED powering is reversed in order.

127. An AED as in claim 122 except wherein the AED additionally comprises a means for manually selecting the priority ordering of all of the potential powering sources for said AED, such that when said manual means is utilized by the user, the user selections override the optionally built in default power priority selection order.

128. An AED constructed within an AED enclosure means, said enclosure means having a plurality of external surfaces whereby one or more of said external surfaces of said enclosure means are each optionally provided with at least one of a decoration, a labeling, and an indicia means for providing the different degrees of visibility required to accommodate the degree of visibility the owner/user desires when said AED is visibly stored awaiting use.

129. An AED as in claim 128 wherein selected surfaces of said AED enclosure means are fitted with means to allow removability and interchangeability of the decoration, labeling, and indicia means including user created decoration, labeling, and indicia means wherein such decoration, labeling, and indicia means includes at least one of text, drawings, images, paintings, icons, photographs and the like.

130. An AED constructed within an AED enclosure means whereby said AED enclosure means being fitted at time of manufacture or refurbishment with frangible external indicia, said frangible external indicia indicating at least one of the date of manufacture, the date of shelf life expiration and hence requirement for refurbishment, and an indication of AED integrity if said frangible external indicia is not breached by prior use or tampering.

131. An AED as in claim 130 wherein said frangible indicia is automatically breached upon opening said AED enclosure means.

132. An AED including an AED enclosure means said enclosure means having multiple outer surfaces wherein all but at least one such outer surface is continuous with its adjacent surfaces and joined to them in such a way as to be non-removable and air tight, and whereby at least one such outer surface is a separable outer surface, being totally separable or hinged at one edge, such that when said separable surface is either totally separated or opened using the hinge, user access is provided to the AED controls and indicators which may optionally be present within,
the improvement being the sealing said removable external surface to its adjacent external surfaces at time of manufacture or refurbishment with a sealing means such that when said surface is sealed to its adjacent surfaces there is formed effectively a closed and sealed AED enclosure that functions as a protective barrier by which water, moisture, and other environmental contaminants are prohibited entry, thus protecting said sealed AED and assuring proper function when needed without fear of environmental contamination or unknown tampering.

133. An AED as in claim 132 wherein said sealed AED additionally provides a means for equalizing the air pressure within the sealed AED with the atmospheric pressure outside said sealed AED.

134. An AED as in claim 132 wherein said sealing means is a gasket of suitable material between the separable and non-separable edges of the surfaces of said AED enclosure.

135. An AED as in claim 132 wherein said sealing means is a strip of tape which bridges the small gap between the separable and non-separable edges of the AED enclosure, said strip of tape being removed prior to AED use.

136. An AED as in claim 132 wherein said sealing means additionally provides a visible indication means to alert the user of any breach of sealing integrity as would occur anytime said enclosure seal was removed whether for use or any other reason.

138. An AED as in claim 136 wherein said visible indication means for indicating breach of said sealing means would additionally contain indicia indicative of at east one of the date of original sealing at time of manufacture or refurbishment and the date of shelf life expiration.

139. An AED enclosed within an outer protective covering means such that water, moisture, and other environmental contaminants are prohibited entry to any AED enclosed within said protective cover means during storage of said AED.

140. An AED as in claim 139 wherein said AED protective covering means is sealed preventing nondisruptive opening of said AED covering means, said seal created using a suitable sealing means such as tape, glue, putty, plastic and the like.

141. An AED as in claim 140 wherein said AED protective covering sealing means is at least one of a heat seal, an adhesive seal, a hook and loop seal, an adhesive seal, a putty seal, a plastic seal, a metal band seal, and a tape seal.

142. An AED as in claim 140 wherein said AED protective covering means additionally provides means for equalizing the air pressure within the sealed AED protective covering means with the atmospheric pressure outside said sealed AED protective covering means.

143. An AED as in claim 140 wherein said sealed AED protective covering means is additionally fitted with a quick opening means such that the user is not appreciably delayed in accessing the AED when needed despite the presence of said sealed covering means protecting said AED from tampering and contamination.

144. An AED as in claim 140 wherein said AED protective covering means totally encloses an AED and with the date of sealing of said AED enclosure means being displayed to an observer by at least one of date printing and date encoding indicating means, said sealed AED covering means and said date indicating means requiring disruption before use of said AED enclosed within said sealed protective AED covering means, wherein disruption of said sealing means provides an indication of at least one of prior use and tampering.

145. An AED protective covering means as in claim 140 wherein said date indicating means indicates the date of expiration of the AED signifying the date beyond which said enclosed AED is no longer guaranteed suitable for use and as such requires refurbishment before use.

146. An AED protective covering means as in claim 139 wherein said covering means additionally comprises a means for rapid removal of said outer covering means.

147. An AED protective covering means as in claim 146 wherein said covering means comprises additionally decoration on its various surfaces with various externally visible indicia of varying obviousness to a casual observer when said AED is visibly stored.

148. An AED protective covering means as in claim 147 wherein said covering means comprises additionally means for optionally removably attaching and retaining on its various external surfaces various externally visible indicia of varying obviousness to a casual observer.

149. An AED constructed within an AED enclosure means said enclosure means having a dated AED sealing means such that moisture and contamination is prohibited entry and such that any of prior use, tampering, and expiration of shelf life is obvious to an observer.

150. An AED as in claim 149 wherein said AED sealing means is an outer covering means which both encloses and protects said AED from water and other contaminants.

151. A substitute AED powering means whereby said substitute AED powering means permits an AED which is designed to utilize internal-integral batteries to be powered by external power sources, said substitute AED powering means comprising a substitute power supply module sized and shaped in at least two of its three dimensions to be the same as the internal-integral battery of an AED and further comprising power connecting electrodes geometrically placed identical to those of the AED's internal-integral batteries, said substitute power supply module optionally replacing said internal-integral battery of any AED utilizing such internal-integral batteries and thus permitting use of external power sources to power AEDs originally designed to use internal-integral batteries.

152. A substitute AED power means as in claim 151, said AED power means further comprising circuitry means for converting said external electrical power to the same nominal voltage and current requirements as originally supplied by the internal-integral battery which said substitute AED power means replaces.

153. A substitute AED power means as in claim 152, said AED power means further comprising a built in battery means for supplying AED power when said substitute AED power means is not connected to a source of said external electrical power.

154. A substitute AED power means as in claim 153, said AED substitute power means further comprising a user accessible and user settable means for establishing priority of power usage when more than one source of power is available to said substitute AED power means.

155. A substitute AED power means as in claim 153, said AED substitute power means further comprising automatic power selection means such that the power source actually used to power the AED is automatically selected according to a pre determined priority.

156. A substitute AED power means as in claim 152, said AED substitute power means further comprising a single power cord for connecting to at least one of external direct current power and external alternating current.

157. A substitute AED power means as in claim 152, said AED substitute power means further comprising two power cords, one for accessing external direct current power and one for external alternating current.

158. A substitute AED power means as in claim 152, said AED substitute power means further comprising one or more external power connectors whereby each said external power connector is designed specifically for a given type of external power and permits said external power source to be removably connected to said substitute AED power means using the appropriate power cords for each potential source of external power, said external power being one or more of direct current power and external alternating current.

159. An AED business method in which AEDs are supplied to customers for a single patient episode of use, said single use AED being sold, leased or rented to said customer.

160. A method of doing AED business as in claim 159, but which further includes a service whereby said single patient episode of use AED may be returned to the factory for refurbishment after a single patient episode of use or expiration of the shelf life of said AED, said refurbishment thus permitting an additional subsequent single patient episode of use, said refurbishment process following said use or said shelf life expiration being repeatable one or more times.

161. A method of doing business as in claim 160, where in said refurbished AED which is returned to the user is an equivalent AED in form and function, but said returned AED is not the identical AED as was sent by the user to the refurbishment center after said single patient episode of use or said shelf life expiration.

162. A method of doing business as in claim 161 where the replacement AED is sent to the customer before the customer returns their AED for refurbishment.

163. A method of doing AED business in which a specified period of AED access is sold to a customer by an AED access and refurbishment provider business entity.

164. A method of doing business as in claim 163 wherein after the specified AED access period has elapsed the customer discards the AED.

165. A method of doing business as in claim 163 wherein after said specified AED access period has elapsed the customer is required to return the AED or said customer is liable for additional charges.

166. A method of doing business as in claim 163 wherein said AED access period is equal to the AED shelf life.

167. A method of doing business as in claim 163 wherein said AED access period is not equal to the AED shelf life.

168. A method of doing business wherein conventional AEDs which are originally designed to be user refurbishable in the field are after use sent to said AED access service and refurbishment center business entity for refurbishment of said conventional AED, said refurbished conventional AED being then returned to the owner thus refurbished and ready for use.

169. A method of doing business as in claim 168 wherein said conventional AED returned to the user is an equivalent conventional AED replacement in both form and function, but not the identical conventional AED as was sent by the user to said refurbishment center.

170. A method of doing business as in claim 169 where said conventional AED replacement is sent to the customer before the owner sends in their conventional AED for refurbishment.
